(19) United States
(12) Patent Application Publication
(10) Pub. No.: US 2015/0259429 A1
(43) Pub. Date: Sep. 17, 2015

(54) CLUSTER OF DIFFERENTIATION 36 (CD36) AS A THERAPEUTIC TARGET FOR HIV INFECTION

(71) Applicants: INSTITUT CURIE, PARIS CEDEX 05 (FR); INSERM (INSTITUT NATIONAL DE LA SANTE ET DE LA RECHERCHE MEDICALE), PARIS CEDEX 13 (FR)

(72) Inventors: Philippe Benaroch, Paris (FR); Stefano Berre, Vincennes (FR)

(21) Appl. No.: 14/424,456

(22) PCT Filed: Aug. 27, 2013
(86) PCT No.: PCT/EP2013/067728

§ 371 Date: Feb. 27, 2015

(30) Foreign Application Priority Data
Aug. 28, 2012 (EP) 12306027.9

(51) Int. Cl. C07K 16/28; C12N 15/113; A61K 39/395; A61K 31/713; G01N 33/68; A61K 45/06

(52) U.S. Cl. CPC C07K 16/2896; G01N 33/6872; A61K 45/06; A61K 39/3955; A61K 31/713; C12N 15/1138; G01N 2333/70596; G01N 2500/10; G01N 2500/04; C07K 2317/24; C07K 2317/35; C07K 2317/622; C07K 2317/54; C07K 2317/626; C07K 2317/76; C12N 2310/11; C12N 2310/14; C12N 2310/12

(57) ABSTRACT

The present invention concerns new methods for treating HIV infection by using CD36 inhibitors, antibodies or conjugates, and methods for identifying new molecules of interest for treating HIV infection.

### FIELD OF THE INVENTION

The present invention relates to the field of medicine and provides new therapeutic agents for treating HIV infection.

### BACKGROUND OF THE INVENTION

Human immunodeficiency 1 (HIV-1), identified in 1983 as the cause of acquired immune deficiency syndrome (AIDS), remains a global health threat responsible for a world-wide pandemic. CD4+ T lymphocytes play a central role in the pathogenesis of human immunodeficiency virus (HIV) infection. In viremic patients, these cells are the major cellular target for the virus: HIV multiplies intensively in this sub population during the acute phase of the disease and continues to propagate in these same cells during the clinical latency phase. Despite many years of potent antiretroviral therapy, latently infected cells and low levels of plasma virus have been found to persist in HIV-infected patients. Highly active anti-retroviral therapy (HAART) is very effective in suppressing HIV-1 replication, thus allowing restoration of the immune system and overall is a lifesaver for infected individuals. However, discontinuation of therapy leads to a rapid rebound in plasma viral load often to pretreatment levels, in as little as two weeks (Davey R T Jr et al, 1999, Proc Natl Acad Sci USA, 96(26): 15109-14). New cellular factors interacting with various steps of the HIV-1 replication cycle, such as entry, integration, transcription, and assembly of new viral progeny have been identified. Interference with innate immune functions, like phagocytosis, autophagy, cytokine production, and T-cell activation by HIV-1 has been found to contribute to virus replication and latency. Growing evidence indicates an important role of infected macrophages in HIV-1-associated diseases. Macrophages are relatively long-lived cells that are less prone to cell death as a result of HIV-1 infection than other cells and are therefore likely to represent a major reservoir of viral particles. With the half-lives of macrophages reported to be of the order of months to years, depending on the specific type of macrophage, and with reports of higher concentrations of antiviral drugs being required to inhibit HIV-1 replication in macrophages than in T cells, infected macrophages pose a serious problem for viral eradication with current antiretroviral therapy.

Most of the viral strains circulating in HIV infected patients are macrophage tropic (CCR5 tropic). Macrophage tropic HIV-1 variants are predominant during the asymptomatic phase and are present during all stages of infection (see intro of Kootstra, N. A., 1994. J Virol. 68:6967-75.). Moreover, macrophages can produce viral particles during extended periods of time as they are resistant to the cytopathic effect of the infection. Infected macrophages are considered as important viral reservoirs because of this resistance and the fact that they store intracellularly large numbers of infectious viral particles. Even under combined antiretroviral therapy (cART), HIV-1 continues to persist in macrophages. Better understanding of HIV-1 infection in macrophages may lead to new adjunctive therapies to improve cART, specifically targeting this viral reservoir and ameliorating tissue-specific diseases. If HIV-1 infection is to be cured, new strategies that will eradicate these reservoirs will be required. Very little data relating to host proteins involved in the HIV viral cycle in macrophage are available. Indeed, no genome-wide screen has been performed so far on such cell type.

Accordingly, there is a significant need for alternative or additional HIV treatments targeting host factors, especially host factors specific of viral reservoirs such as macrophages, in order to avoid immune viral escape.

### SUMMARY OF THE INVENTION

The present invention provides new therapeutic agents for treating HIV infection. The inventors have shown that silencing of CD36 expression in HIV-1-infected primary macrophages efficiently reduces the release of HIV-1 particles. They have further shown that antibodies specific for CD36 are actively and specifically transported by infected macrophages into their Virus-Containing Compartment (VCC) at 37° C. Importantly, exposure of HIV-1-infected macrophages to antibodies specific for CD36 for 24 h led to strong reductions of the released viral particles which accumulate instead intracellularly. They have demonstrated that exposure to bivalent antibodies specific for CD36 induces virions tethering at the site of viral assembly, i.e. in the VCCs of macrophages. Since CD36 expression is restricted to very few type of cells, these results open the door for new therapies to target viral reservoirs constituted by infected macrophages.

Therefore in a first aspect, the present invention concerns a composition comprising an antibody that recognizes and binds CD36, or an antigen-binding fragment or derivative thereof, a CD36 antagonist, or a conjugate comprising at least one CD36 targeting agent associated to at least one anti-HIV effector moiety, and a pharmaceutically acceptable excipient, for use in the treatment of HIV infection.

The composition may comprise a CD36 antagonist. Such CD36 antagonists may be selected from the group consisting of antibodies or aptamers which bind to CD36 or fragments thereof; soluble ligands which bind to CD36 or fragments thereof; soluble CD36 which bind to its ligands; fusion polypeptides, peptides, small molecules, peptidomimetics inhibiting the CD36 activity; and nucleic acid molecules interfering specifically with CD36 expression. Such CD36 antagonist is preferably an antagonist which, preferably specifically, recognizes and binds to a CD36 molecule or fragment thereof, and is preferably selected from the group consisting of an antibody or an aptamer which specifically recognizes and binds to CD36 or a fragment thereof, a nucleic acid molecule interfering specifically with CD36 expression, and a small molecule inhibiting the CD36 activity. More preferably, said CD36 antagonist is a function-blocking monoclonal antibody against CD36 or a nucleic acid molecule interfering specifically with CD36 expression.

In one particular embodiment, said CD36 antagonist is a nucleic acid molecule interfering specifically with the expression of CD36. This nucleic acid molecule is preferably selected from the group consisting of a single-stranded polynucleotide sequence (either RNA or DNA) capable of binding to target mRNA (sense) or DNA (antisense) sequences, an interfering RNA (iRNA), and a ribozyme, more particularly selected from the group consisting of a RNAi, an antisens nucleic acid and a ribozyme. More preferably, this nucleic acid molecule is a RNAi, even more preferably a siRNA. In particular, the this nucleic acid molecule may be a siRNA selected from the group consisting of siCD36_{—}1 of SEQ ID No.1 (GAACCUAUUGAUGGAUUAATT), siCD36_{—}3 of SEQ ID No.2 (CCUUCACUAUCAGUUGGAATT) and siCD36_{—}4 of SEQ ID No.3 (GCAACAUUCAAGUUAAGCATT), and any combination thereof. In another particular embodiment, said CD36 antagonist is selected from the group consisting of an antibody which specifically recognizes and binds to the CD36 receptor or a fragment thereof, and antibody which specifically recognizes and binds to CD36 ligand LDLox or to a fragment thereof, said antagonist being preferably a neutralizing antibody. Said CD36 antagonist is preferably an antibody which specifically recognizes and binds to the CD36 receptor or a fragment thereof, even more preferably, a function-blocking monoclonal antibody against CD36.

In still another embodiment, said CD36 antagonist is a small molecule selected from the group consisting of salvianolic acid B, rosmarinic acid, sodium danshensu, 3-cinnamoyl indole, 13 pentyl berberine, hexarelin, nanoblockers, statins or antioxidants such as α-tocopherol and SS peptides, Sulfo-N-succinimidyl oleate and Ursolic acid, and any combination thereof. In one further embodiment, the CD36 antagonist is a small molecule selected from the group consisting of Sulfo-N-succinimidyl oleate (SSO), Ursolic acid, AP5258 (Arteria), and any combination thereof.

Alternatively, the composition may comprise an antibody that recognizes and binds CD36, or an antigen-binding fragment or derivative thereof. Preferably, said antibody or antigen-binding fragment or derivative thereof, is directed against the extracellular domain of CD36. Said antibody may be a full-length antibody. Preferably, said antibody is a monoclonal antibody. The antibody may be of the IgG, IgE or IgD type, preferably of the IgG type. The antibody may be a humanized, chimeric or human antibody. The antibody may also be camelid heavy-chain antibody, and in particular humanized camelid heavy-chain antibody. Preferably, said antibody, or antigen-binding fragment or derivative thereof, is bivalent. In particular, the antigen-binding fragment is selected from the group consisting of F(ab′)2 di-scFvs, sc(Fv)₂ fragment, (VHH)₂ fragment and diabody.

In another alternative, the composition comprises a conjugate comprising at least one CD36 targeting agent associated to at least one anti-HIV effector moiety. Preferably, said CD36 targeting agent is an antibody recognizing and binding to CD36, or to a fragment thereof. Preferably, said anti-HIV effector moiety is a HIV co-receptor or an anti-HIV drug. In particular, the HIV effector moiety may be a HIV co-receptor selected from the group consisting of CXCR4, CCR5, CD4, T20, NBD-556, CD4M33, fragments thereof and functional equivalents thereof, preferably selected from the group consisting of CCR5, CXCR4, CD4, T20, NBD-556, and CD4M33. The HIV effector moiety may also be an anti-HIV drug selected from the group consisting of reverse transcriptase inhibitors, integrase inhibitors and protease inhibitors. In a particular embodiment, the HIV effector moiety is a reverse transcriptase inhibitor, preferably selected from the group consisting of deoxythymidine, zidovudine, stavudine, didanosine, zalcitabine, abacavir, lamivudine, emtricitabine, entecavir, apricitabine, adefovir, efavirenz, nevirapine, delavirdine, etravirine, rilpivirine and tenofovir. In another particular embodiment, the HIV effector moiety is an integrase inhibitor, preferably selected from the group consisting of raltegravir, elvitegravir and MK-2048. In a further particular embodiment, the HIV effector moiety is a protease inhibitor, preferably selected from the group consisting of lopinavir, indinavir, nelfinavir, amprenavir, ritonavir, saquinavir, atazanavir, fosamprenavir, tipranavir and darunavir.

The composition of the invention may be used in combination with another anti-viral treatment such as HAART. The composition may be administered before, during or after said anti-viral treatment. In particular, the composition may be used in combination with an anti-HIV drug administered before, during or after HAART. In a particular embodiment, the composition is used in combination with HAART. HAART may comprise one or several reverse transcriptase inhibitors, one or several integrase inhibitors, and/or one or several protease inhibitors. HAART preferably comprises one or several reverse transcriptase inhibitors and/or one or several protease inhibitors, more preferably one or several reverse transcriptase inhibitors and one or several protease inhibitors.

In a second aspect, the present invention provides a conjugate comprising at least one CD36 targeting agent associated to at least one anti-HIV effector moiety/molecule, optionally associated through a linker, optionally via recombinant linking or chemical conjugation. In a preferred embodiment, said CD36 targeting agent is a CD36 antagonist. In another preferred embodiment, said CD36 targeting agent is an antibody recognizing, preferably specifically, and binding to CD36, or to a fragment thereof.

In a preferred embodiment, said anti-HIV effector moiety/molecule is an anti-HIV drug, preferably used in HAART, or a HIV co-receptor.

Preferably, the HIV co-receptor is selected from the group consisting of CCR5, CXCR4, CD4, T20, NBD-556, CD4M33, fragments thereof and functional equivalents thereof, more preferably selected from the group consisting of CCR5, CXCR4, CD4, T20, NBD-556 and CD4M33, even more preferably selected from the group consisting of CCR5, CXCR4, CD4 and T20.

Preferably, the anti-HIV drug is selected from the group consisting of reverse transcriptase inhibitors, integrase inhibitors and protease inhibitors. In a particular embodiment, the anti-HIV drug is a reverse transcriptase inhibitor, preferably selected from the group consisting of deoxythymidine, zidovudine, stavudine, didanosine, zalcitabine, abacavir, lamivudine, emtricitabine, entecavir, apricitabine, adefovir, efavirenz, nevirapine, delavirdine, etravirine, rilpivirine and tenofovir. In another particular embodiment, the anti-HIV drug is an integrase inhibitor, preferably selected from the group consisting of raltegravir, elvitegravir and MK-2048. In a further particular embodiment, the anti-HIV drug is a protease inhibitor, preferably selected from the group consisting of lopinavir, indinavir, nelfinavir, amprenavir, ritonavir, saquinavir, atazanavir, fosamprenavir, tipranavir and darunavir.

In a third aspect, the present invention concerns a method for screening for compounds useful for the treatment of HIV infection, said method comprising: determining whether a candidate compound inhibits the activity or expression of CD36, wherein the ability of said candidate compound to inhibits the expression or activity of said CD36 is indicative of the usefulness of said candidate compound for the treatment of HIV infection.

The screening method according to the invention may further comprise testing the candidate compound in a cellular model, such as DC/macrophage/monocyte cell lines infected with HIV, or administering the candidate compound to a non-human animal model of HIV infection, such as Rhesus Monkeys or SIVmac239.

The present invention also concerns a method for screening for compounds useful for the treatment of HIV infection, said method comprising determining whether a candidate compound recognizes and binds to CD36, or reduces/inhibits expression of CD36, wherein the ability of said candidate compound to recognize and bind to CD36 or to reduce/inhibit expression of CD36 is indicative of the usefulness of said candidate for the treatment of HIV infection.

### DESCRIPTION OF THE FIGURES

FIG. 1. Among several lipoprotein receptors, CD36 is the only one to be present into the VCC

(FIG. 1A) Confocal sections of HIV-1-infected macrophages stained for the indicated markers at day 7 post-infection. PC: Pearson Coefficient. Bars, 10 μm. (FIG. 1B) Immuno-EM of HIV-1-infected macrophages. Ultrathin cryosections were double labeled for p17 Gag with PAG10 and for CD36 with PAG15. (FIG. 1C) Quantification of the co-distribution of Gag with various LDL receptors imaged in (FIG. 1A). Geo mean and their standard deviation are presented.

FIG. 2. Plasma membrane connection of CD36+ compartments in infected and uninfected macrophages

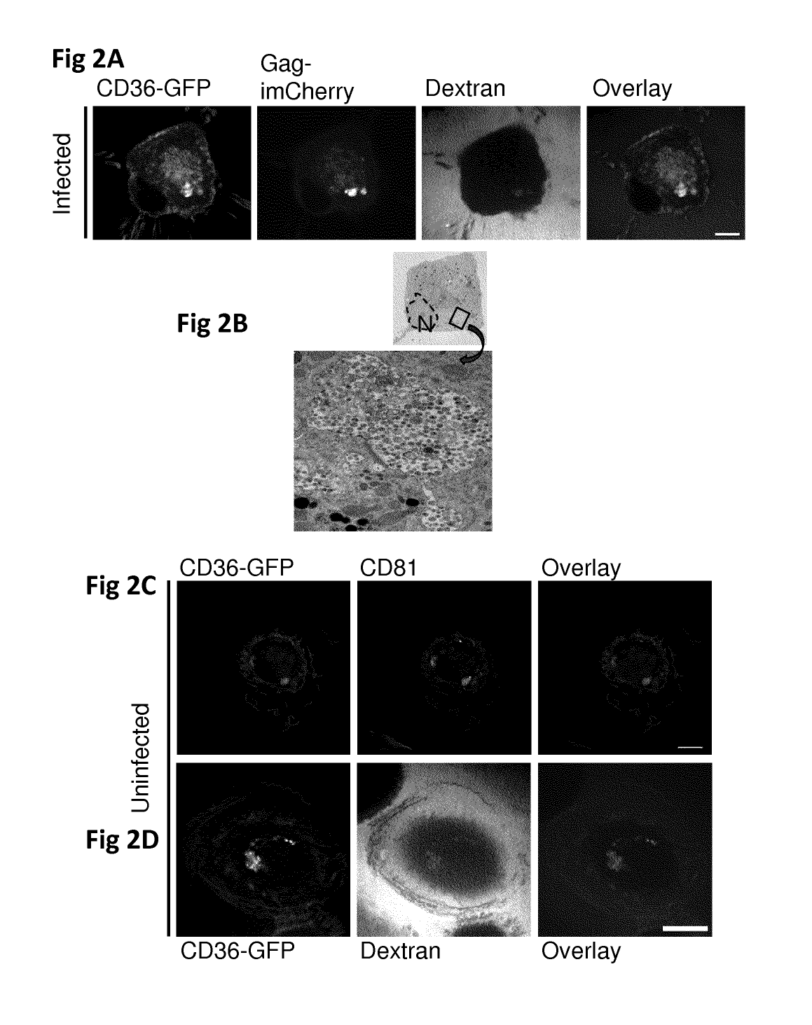

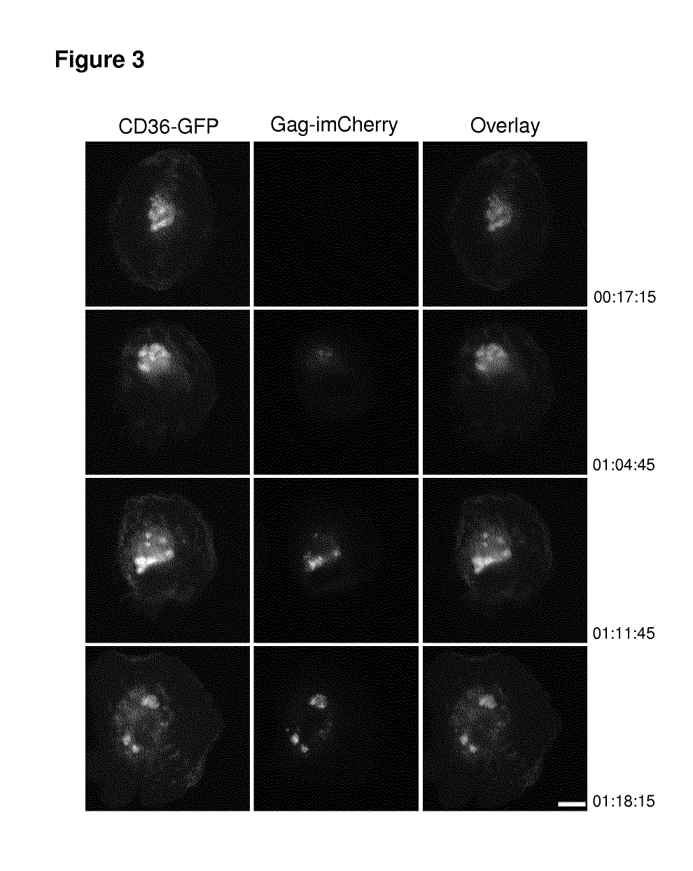

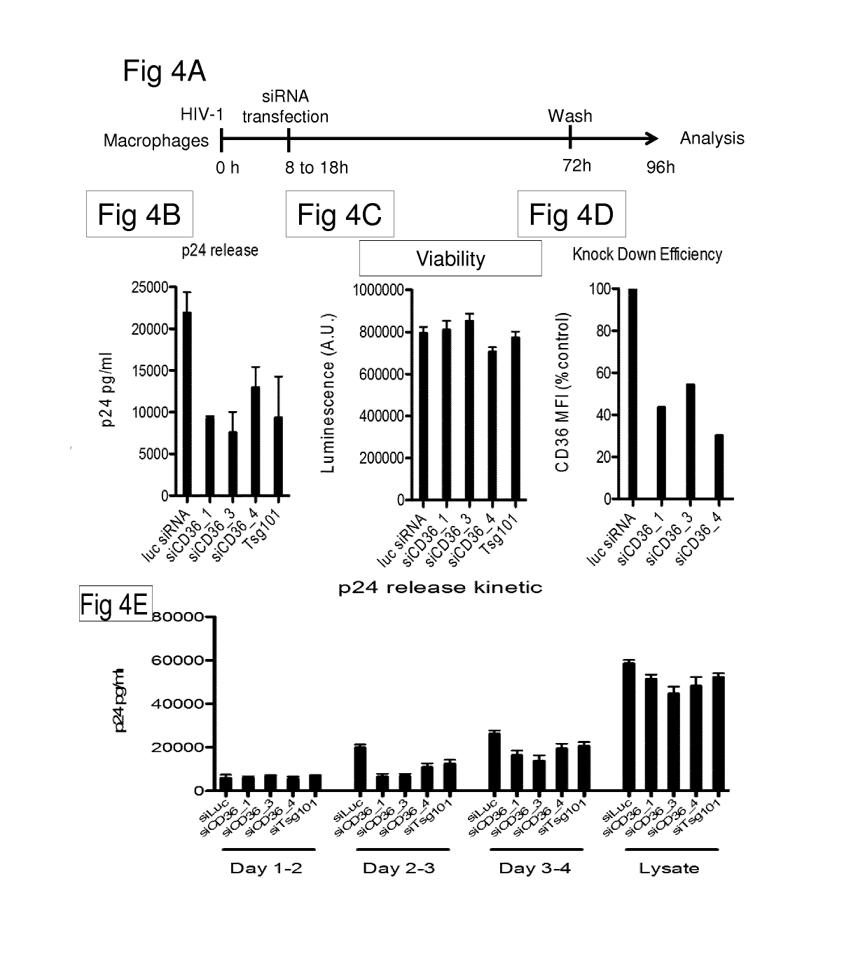

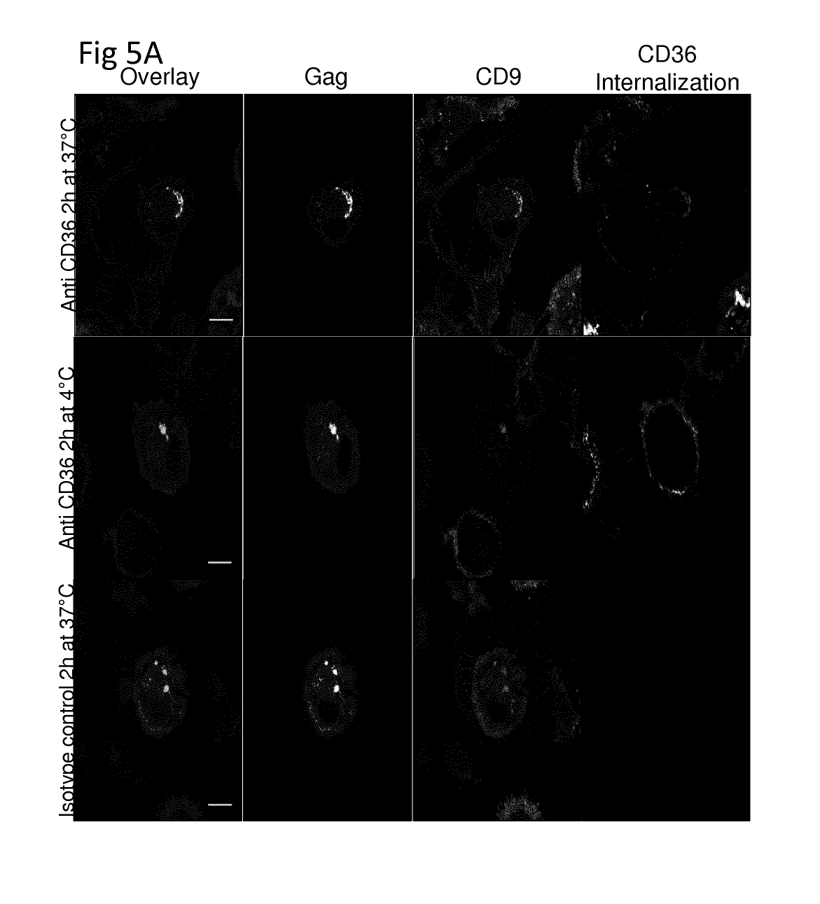

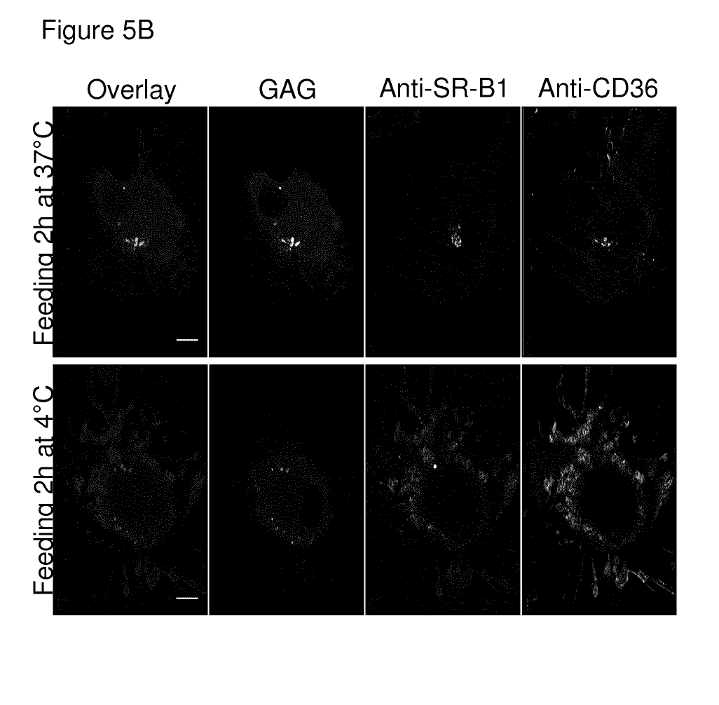

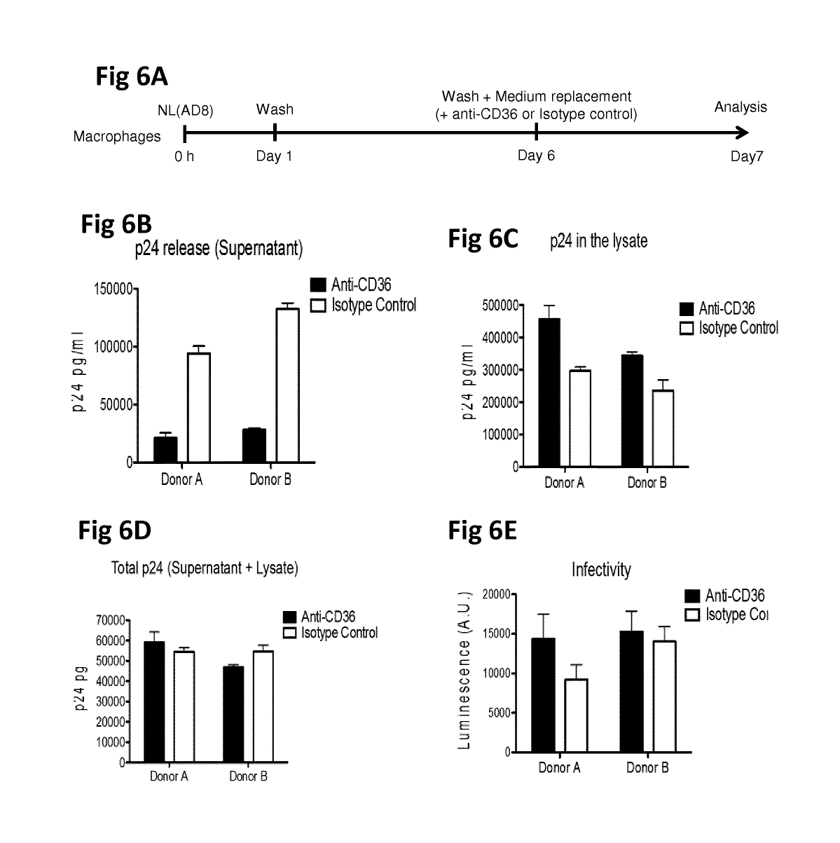

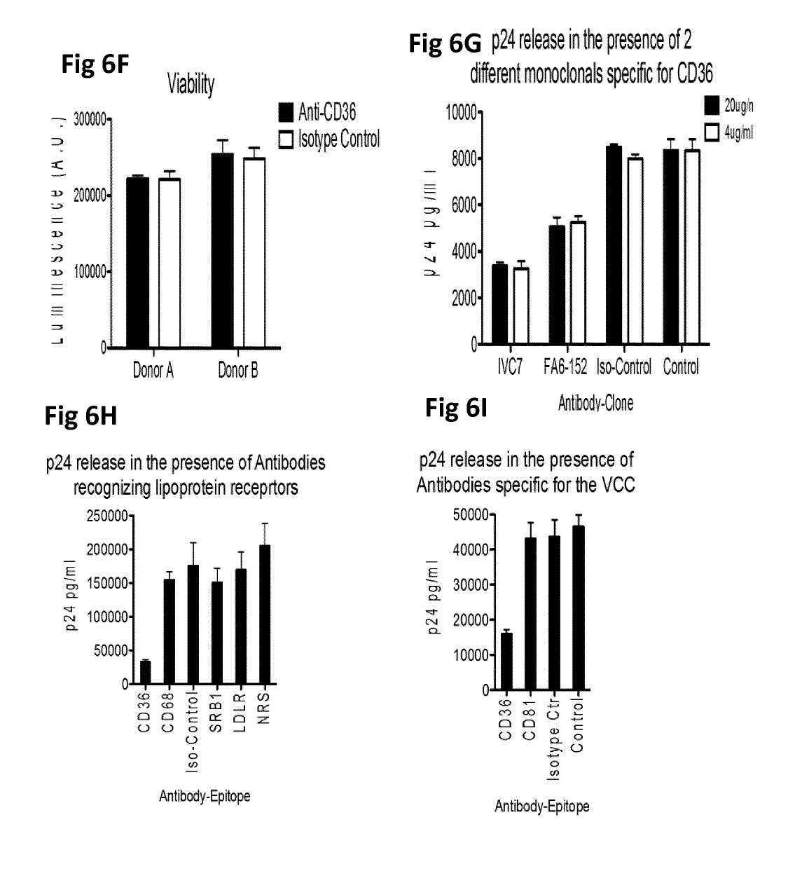

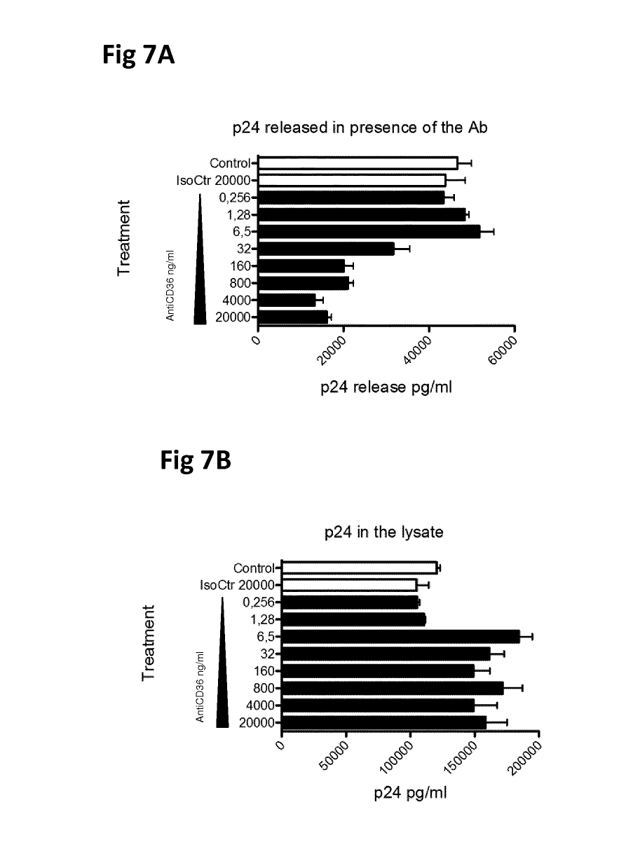

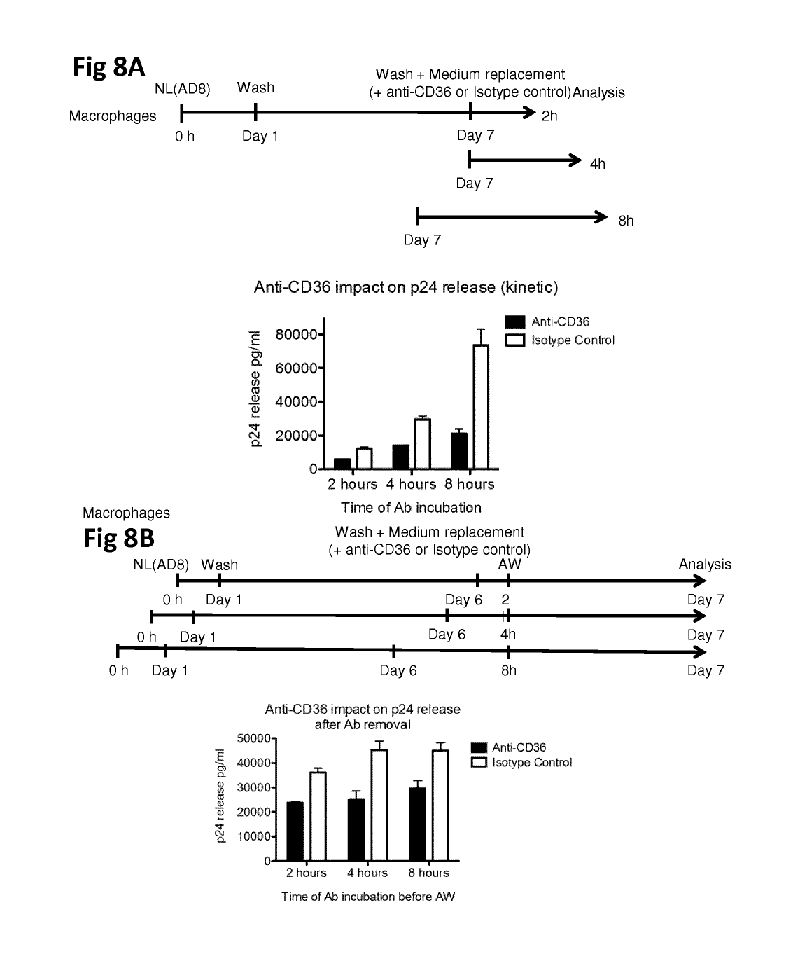

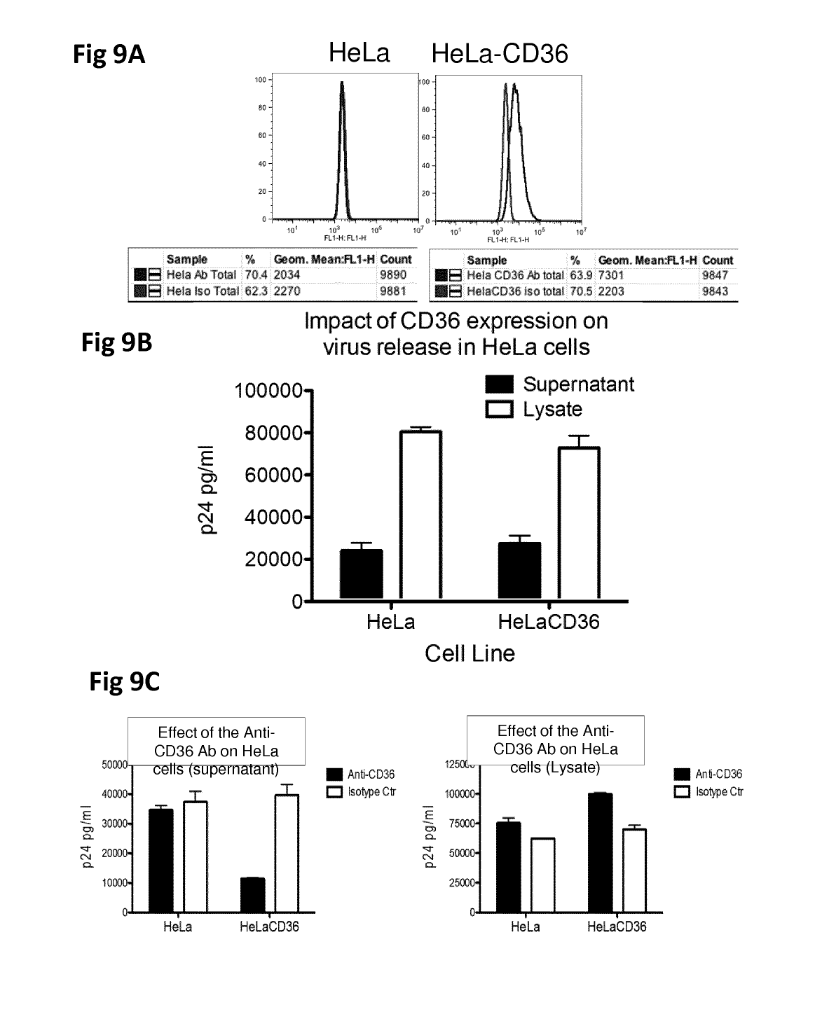

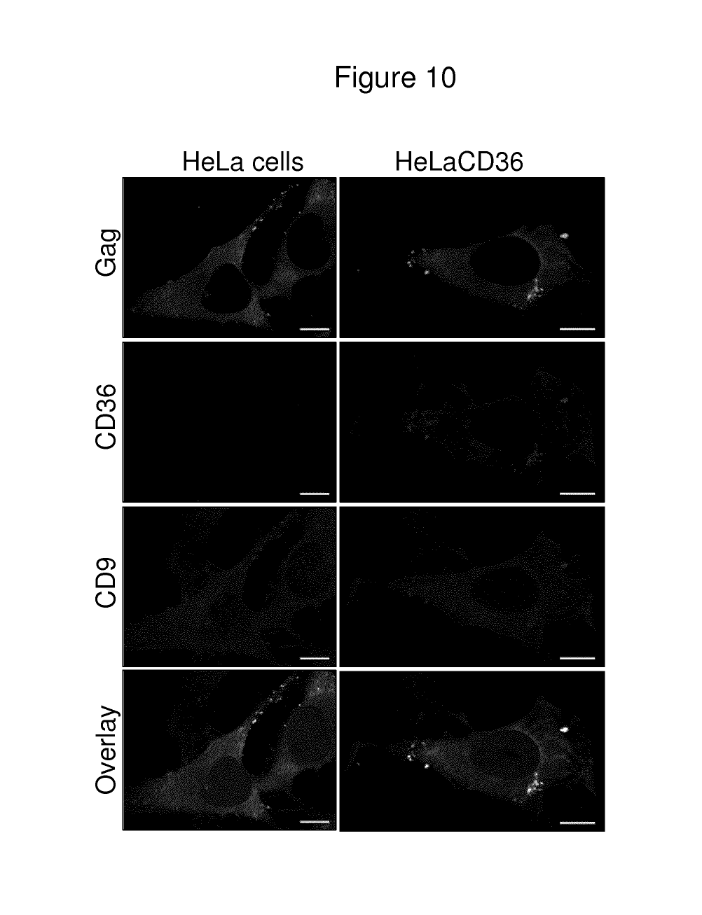

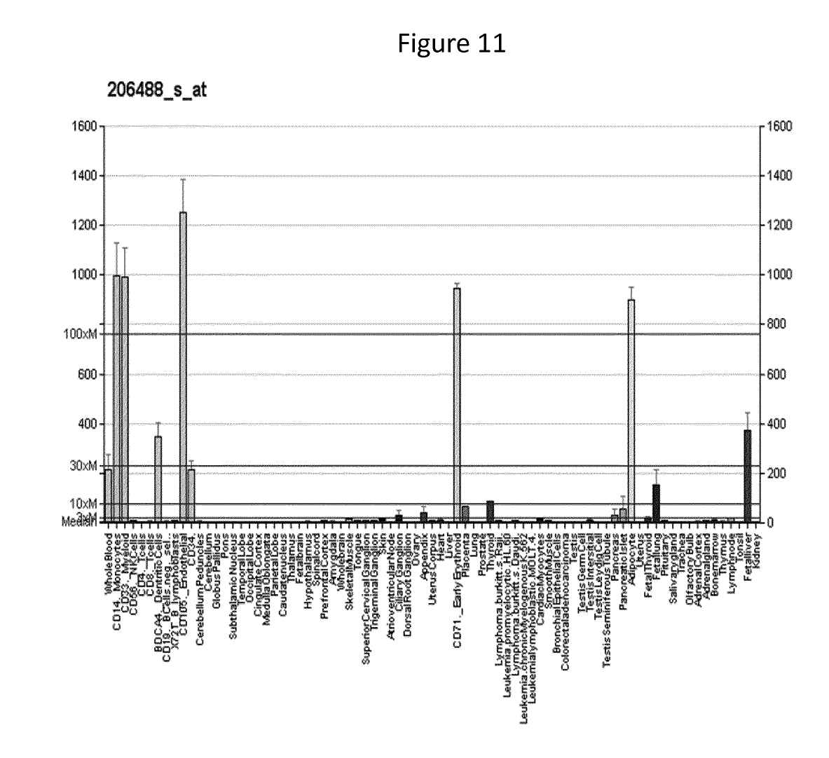

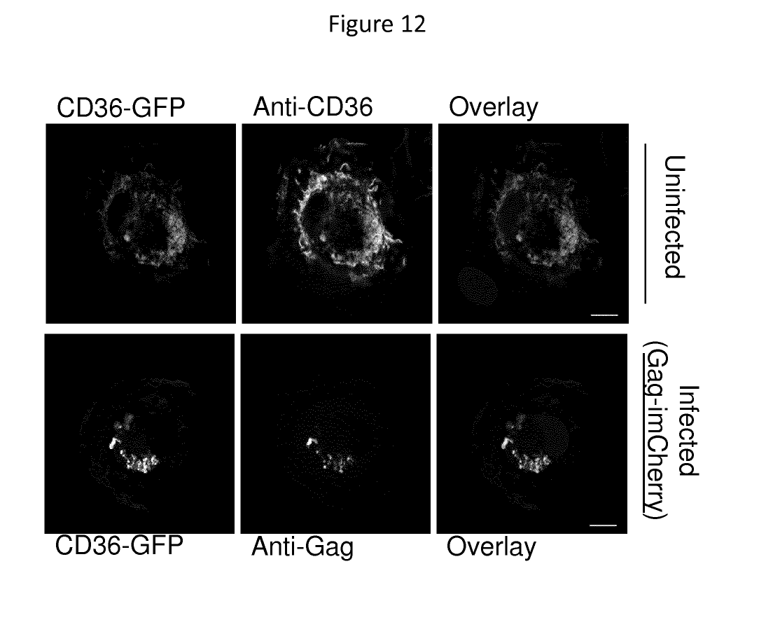

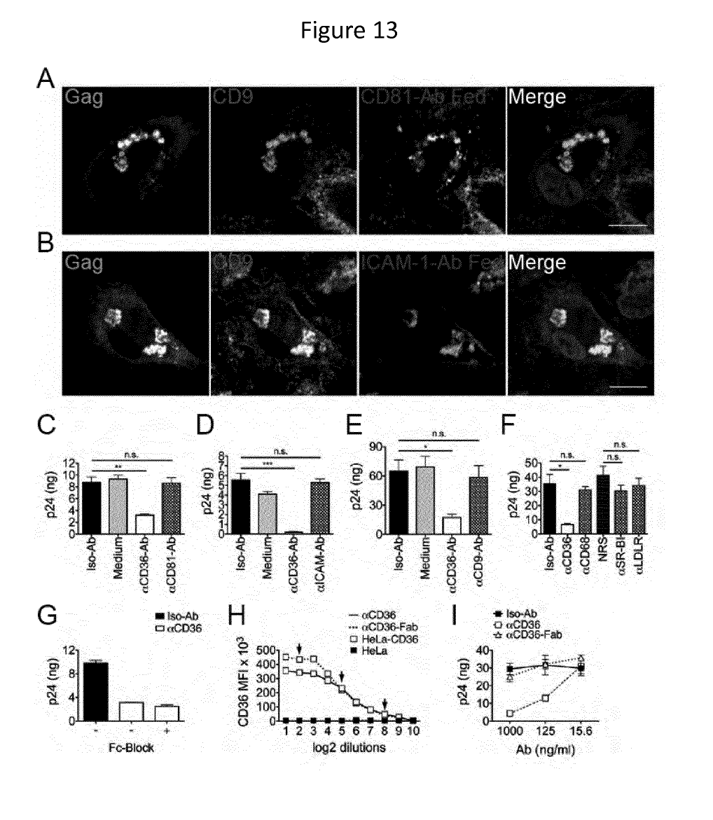

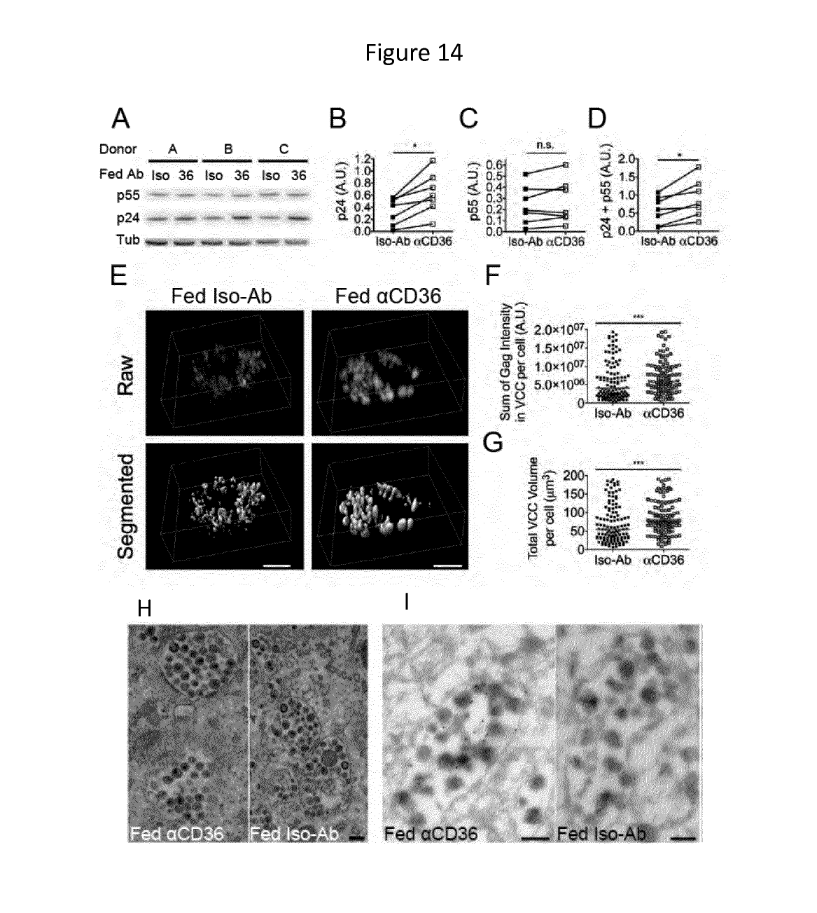

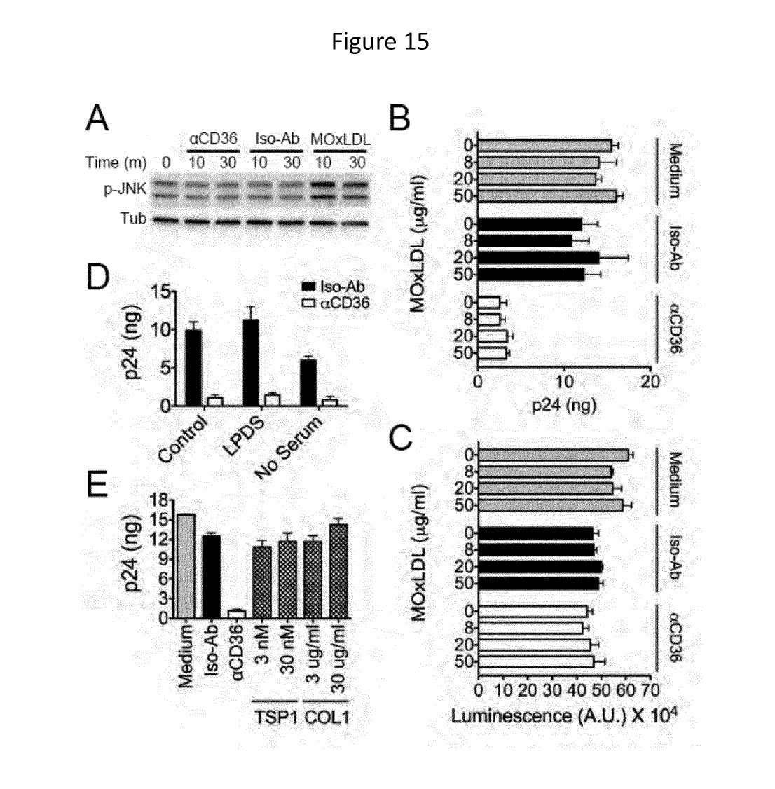

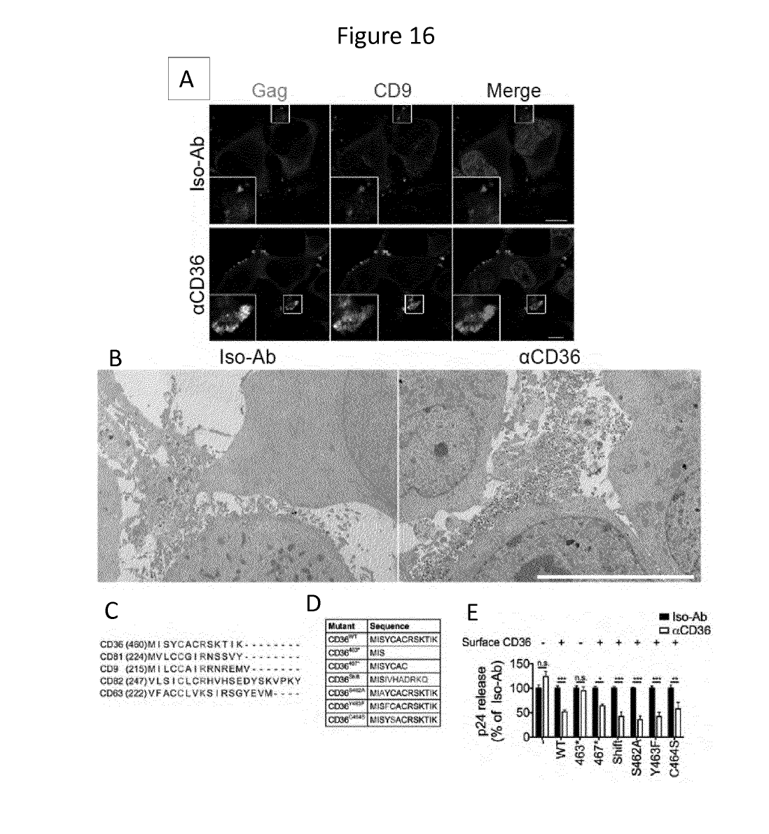

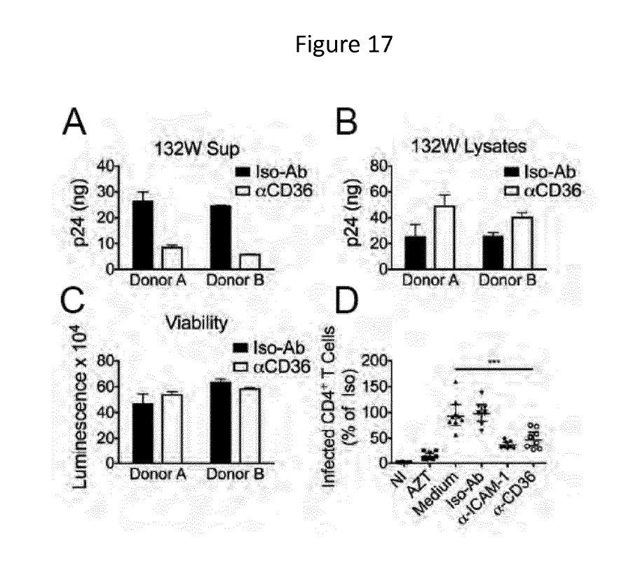

(FIG. 2A) Correlative fluorescent and electron microscopy. Primary macrophages grown on coverslips with coordinates were infected with a lentiviral vector encoding CD36-GFP together with Vpx VLP and 5 days later with HIV Gag-imCherry. At day 7 p.i. cells were exposed to a 10 kD dextran-Alexa 647 and immediately after imaged by spinning disk microscopy to follow the indicated markers. The same coverslips were then embedded in epon and processed for EM. An overview of the macrophage imaged by EM is presented on the right panel. N; nucleus. (FIG. 2B) Magnification of the region boxed on the right panel, with viral budding profiles visible at the limiting membrane and both mature and immature viral particles. (FIG. 2C) Confocal microscopy of primary macrophages infected with a lentiviral vector encoding CD36-GFP for 5 days, fixed and stained for CD81. (FIG. 2D) As in (FIG. 2A), here macrophages infected with the lentiviral vector encoding CD36-GFP for 12 days were exposed to dextran-Alexa 647 and immediately after imaged by spinning disk microscopy.

FIG. 3. Biogenesis of the Gag+CD36+ compartment in HIV-1-infected macrophages

Macrophages were first transduced with the lentivector encoding CD36-GFP and 4 days later infected with HIV Gag-iCherry. After another 8 hours, cells were washed, and then live imaged during 2 days. Here are presented images acquired at the indicated time (d:h:m).

FIG. 4. CD36 expression is critical for efficient HIV-1 release by primary macrophages

(FIG. 4A) Schematic representation of the experimental design.

(FIG. 4B) p24 Gag released in the supernatant of macrophages that had been infected with HIV-1 and transfected with the indicated siRNA. After washes at day 3, supernatant were collected 24 h after and analyzed by ELISA for their p24 content.

(FIG. 4C) Cytotoxicity was determined at the end of the experiments using the cell titer glow.

(FIG. 4D) Silencing efficiency was estimated by flow cytometry analysis of the various cell populations that were fixed and stained for CD36. MFI is expressed as % of the control (cells transfected with siRNA specific for luciferase).

(FIG. 4E) Kinetics of the effect of the silencing on the p24 release. Macrophages were infected and transfected as indicated in (FIG. 4A). p24 dosage of their supernatant harvested at the indicated time periods and of the different cell lysates performed at day 4 is presented.

FIG. 5. CD36-specific Abs are specifically internalized into the VCC

(FIGS. 5A and B) Confocal micrographs of HIV-1-infected macrophages are presented. (FIG. 5A) Cells were exposed for 2 hours either at 37° C. or at 4° C. with the indicated mAb. Cells were then fixed and stained for CD9 and Gag, whereas isotype control mAbs as well as CD36 specific mAb were revealed with appropriate secondary antibodies.

(FIG. 5B) Experimental set up was similar to (FIG. 5A) excepted that antibodies specific for SRB1 (rabbit antibodies) and CD36 (mouse mAb) were simultaneously added to the infected cells for 2 hours. Both types of antibodies are internalized at 37° C. (not at 4° C.) but only the CD36-specific mAb co-localizes with Gag+ compartments.

FIG. 6. CD36-specific antibodies modulate HIV-1 release from macrophages

(FIG. 6A) Schematic representation of the experimental design.

(FIG. 6B) Dosage of p24 Gag in the 24 hr culture supernatants harvested as indicated in FIG. 6A, and (FIG. 6C) in the corresponding cell lysates. (FIG. 6D) Total p24 Gag found in the supernatant+in the cell lysates. (FIG. 6E) Infectivity of the virions on the reporter cell line TZM-bl produced by the macrophages subjected to the indicated Ab treatment was evaluated using the same amount of p24 (2 ng), (see Materials and methods). (FIG. 6F) Cell viability was measured at day 7 p.i. using the CellTiter glo kit. (FIG. 6G) Comparison of the effects of two different CD36-specific mAbs (IVC7 and FA6-152), and of their isotype control (Iso-control), at the two indicated concentrations on the p24 Gag release. Control, untreated cells. (FIG. 6H) Comparison of the effects of antibodies specific for different lipoprotein receptors on the p24 Gag release. NRS, normal rabbit serum. (I) Comparison of the effects of mAbs specific for CD36 and CD81 (a tetraspanin associated with VCCs) on the p24 Gag release.

FIG. 7. Titration of the effect of CD36-specific mAb on HIV-1-infected macrophages.

HIV-infected macrophages were treated with medium containing different concentrations of the IVC7 mAb or isotype control. After 24 hours the p24 was quantified in the supernatant and into the cell lysate.

FIG. 8. Kinetics and duration of the impact of the CD36-specific mAb exposure on p24 release

(FIG. 8A) Schematic representation of the experimental design. Dosage of p24 Gag in the supernatant harvested after mAb exposure for 2, 4 or 8 hours. (FIG. 8B) Schematic representation of the experimental design. Dosage of p24 Gag in the supernatant harvested after mAb exposure for 2, 4 or 8 hours, followed by acid wash (AW) and re-incubation for 24 hours.

FIG. 9. Characterization of HeLa-CD36

(FIG. 9A) Flow cytometry profiles of HeLa and HeLa-CD36 cells stained with a mAb specific for CD36 (blue) or its isotype control (red). (FIG. 9B) p24 release from HeLa and HeLa-CD36 cells infected with HIV-1 for 2 days. (FIG. 9C). HeLa and HeLa-CD36 cells were HIV-1 NL(AD8)-infected for two days, washed and exposed for 24 hours to the indicated mAb. Dosage of p24 Gag was performed in the supernatant and in the cell lysates (performed at day 2 p.i.)

FIG. 10. CD36 expression in HeLa cells induces the formation of VCC-like structures

Confocal sections of HeLa cells expressing or not CD36, HIV-1 infected for 3 days and immunostained for the indicated markers

FIG. 11. Graph showing the tissue specific profile of the CD36 expression

Data were obtained from BioGPS website and refers to the mRNA levels

FIG. 12. Distribution of CD36-GFP in HIV Gag-imCherry-infected and non-infected macrophages

Confocal sections of primary macrophages infected with CD36-GFP lentiviral vector alone or with HIV-1 Gag-imCherry. HIV-1-infected cells were fixed and stained for the indicated markers. Bar, 10 μm.

FIG. 13. The anti-CD36-mediated inhibition of HIV-1 release is specific and requires bivalent binding.

(FIGS. 13A and B) Exogenous CD81 and ICAM-1-specific antibodies are transported to the VCC. Macrophages were infected with HIV NLAD8 for 3 days, washed and incubated for 2 hr with (FIG. 13A) a CD81-specific mAb or (FIG. 13B) an ICAM-1-specific mAb. mAbs were used at 5 μg/ml final. Cells were then washed, fixed, permeabilized and stained for Gag and CD9 to identify VCCs. Bars, 10 μm. (FIGS. 13C, D, E and F) Quantification of p24 Gag released from macrophages treated overnight with the indicated antibodies. Of note, other antibodies transported to the VCC (FIGS. 13C and 13D), antibodies specific for markers of the VCC (FIGS. 13C, D and E) and antibodies specific for other LDL receptors (FIG. 13F) did not modulate HIV-1 release from macrophages. (FIG. 13G) Quantification of the p24 Gag produced by HIV-1-infected primary macrophages treated with the FA6-152 antibody in the presence or not of Fc-Blocking antibodies. (FIG. 13H) Flow cytometry titration of the anti-CD36 mAb CLB-IVC7 and its Fab fragment. HeLa-CD36 (open symbols) and HeLa cells (black symbols) were stained with serial dilutions of the anti-CD36 mAb and its Fab revealed by appropriate secondary antibodies. MFIs are plotted as a function of the Ab dilutions. (FIG. 13I) Quantification of p24 Gag released from macrophages treated overnight with the anti-CD36 mAb, its Fab fragment or the isotype control. Cells were exposed to the 3 dilutions indicated by arrows on panel (FIG. 13H). Data are shown as mean±SEM of triplicates. In FIG. 13C to FIG. 13I are shown representative experiments. All the experiments have been reproduced at least two times with different donors. One way ANOVA with Tukey's Multiple Comparison Test was used as statistic test (* p≦0.05, ** p≦0.01, *** p≦0.001).

FIG. 14. Exposure to CD36-specific antibodies induces intracellular accumulation of virions in VCCs.

(FIG. 14A) Immunoblot analysis of Gag polypeptides present in lysates of infected macrophages exposed to the CD36 mAb (36) or its isotype control (Iso). Bands corresponding to p55 Gag (p55), p24 Gag (p24) and tubulin (Tub) are indicated. (FIG. 14B to D) Quantifications of the intensity of the various Gag polypeptides present in cell lysates were performed by immunoblot analysis like in (FIG. 14A). The experiments were repeated with cells from 7 donors. p55 Gag and p24 Gag signals were normalized for α-tubulin. Statistical analyses were performed using the Wilcoxon matched pairs test (* p≦0.05). (FIG. 14E) Examples of 3D reconstructions obtained before and after segmentation of image stacks acquired by confocal microscopy of macrophages infected with HIV-1 Gag-iGFP for 7 days, and exposed from day 5 to 7 to the anti-CD36 mAb or its isotype control. Bars, 7 μm. Segmented reconstructions as seen in (FIG. 14E) were quantified using Imaris software. Graphs illustrating the variation of (FIG. 14F) the total Gag intensity per cell and (FIG. 14G) the total volume of VCC per cell are presented. Effect of the anti-CD36 on p24 Gag release was estimated in parallel by Elisa in the 3 donors used for these experiments. The average inhibition of p24 Gag release was of 90%. Data are presented with the median in red. Each square corresponds to a cell. Statistical analyses were performed using the Mann-Whitney test (*** p≦0.001). (FIGS. 14H and I) Electron micrographs of infected macrophages treated with the indicated antibodies. (FIG. 14H) HIV-1 NLAD8-infected macrophages at 6 days pi were washed and treated with anti-CD36 mAbs or with its isotype control for 48 hr. Cells were then fixed, embedded in Epon and processed for EM. Bar, 200 nm. (FIG. 14I) HIV-1 infected macrophages treated as in (H) were prepared for immuno-EM. mAbs present in internal compartments were detected with appropriate secondary antibodies revealed by PAG10. Bars, 200 nm.

FIG. 15. Modulation of HIV-1 release by anti-CD36 antibody exposure is not related to CD36 interaction with MOxLDL, TSP-1 or type-I collagen

(FIG. 15A) Immunoblot analysis of the phosphorylation of JNK after different stimuli. HIV-1 NLAD8 infected macrophages starved for 2 hr in serum-free medium and were treated with anti-CD36 mAb, its isotype control (1 μg/ml) or MOxLDL (at 50 μg/ml) for the indicated period of time. α-Tubulin contents on the same immunoblot are presented for control of loading. (FIG. 15B) Macrophages infected with HIV-1 NLAD8 for 7 days, were washed and pre-treated for 30 min with medium supplemented or not with FA6-152 mAb or its isotype control at 2 μg/ml. Then, medium containing or not MOxLDL was added directly onto the cells to obtain the indicated final concentration of MOxLDL and to keep mAb final concentration at 1 μg/ml. Measure of the p24 Gag released during overnight treatment are presented. (FIG. 15C) Cell viability at the end of the experiment shown in B was measured with the CellTiter-Glo kit. (FIG. 15D) Macrophages at 7 days pi starved for 2 hr in serum free medium and were treated with the anti-CD36 or with the isotype control mAb in complete medium (Control), medium supplemented with Lipoprotein Deficient Serum (LPDS), or in serum-free medium (No Serum). (FIG. 15E) Quantification of the p24 Gag released from HIV-1-infected macrophages treated overnight with the anti-CD36 antibody (FA6-152 at 2 μg/ml), its isotype control or thrombospondin-1 (TSP-1) and type-I collagen (COL1) at the indicated concentrations. Representative experiments are shown. Results have been obtained at least three times from different donors. Data are presented as mean±SEM of triplicates.

FIG. 16. Anti-CD36 antibody treatment induces HIV-1 tethering at the plasma membrane of HeLa cells expressing CD36

(FIG. 16A) Confocal sections of HeLa and HeLa-CD36 cells infected with HIV-1 Gag-iGFP, treated with the anti-CD36 mAb or its isotype control (Iso-Ab) for 48 hr and stained for CD9. Insets are enlargements of the boxed regions. Bars: 10 μm. (FIG. 16B) Electron micrographs of HIV-1 NLAD8-infected HeLa-CD36 cells treated with FA6-152 mAb or its isotype control. Bar, 10 μm. (FIG. 16C) Multiple alignment illustrating the conserved residues present in the cytoplasmic tails of the proteins indicated. Figure adapted from (Primo et al., 2005). (FIG. 16D) Table showing the CD36 mutants produced with the corresponding changes in the C-tail sequence. The mutated amino acids are shown in red. (FIG. 16E) Quantification of p24 Gag in the supernatant of HeLa cell lines stably expressing the indicated CD36 mutants, treated with the anti-CD36 mAb or its isotype control. CD36 cell surface expression was assessed by FACS (indicated by + or −). With the exception of the 467* and the C464S mutants (repeated in 2 independent experiments) data are presented as the means and SEM of three independent experiments performed on three different preparations of stable cell lines. Statistical analysis was performed by two-way ANOVA with Bonferroni's post hoc test (*p≦0.05, **p≦0.01, ***p≦0.001).

FIG. 17. Effect of anti-CD36 antibody exposure on macrophages infected with a primary isolate and on macrophage-to-T cell transmission.

(FIGS. 17A and B) Macrophages from 2 donors were infected with the primary HIV-1 isolate 132W and treated at day 7 pi with anti-CD36 mAb or isotype control. p24 Gag contents were measured in culture supernatant (FIG. 17A) and in corresponding cell lysates (FIG. 17B). (FIG. 17C) Cell viability measured at the end of the experiment with the CellTiter-Glo kit. Data are presented as mean±SEM of triplicates. (FIG. 17D) Macrophages-to-T cells HIV-1-transmission after co-culture of heterologous, activated primary CD4+ T cells with HIV-1-infected macrophages in the indicated conditions. % of infected CD4+ T cells after co-culture was measured by intracellular FACS staining of Gag. Data presented were obtained from 3 independent experiments performed on cells from 6 different donors and are shown as Geo Mean with 95% CI. One-way ANOVA with Tukey's Multiple Comparison Test was used as statistic test (*** p≦0.001).

### DETAILED DESCRIPTION OF THE INVENTION

HIV-1-infected macrophages produce and accumulate newly formed viral particles into intracellular Virus-Containing Compartment (VCC). Analysis of the expression profile of CD36 reveals that it is essentially limited to macrophages, adipocytes, endothelial cells, platelets and dendritic cells.

The inventors have demonstrated, in a surprising way, that CD36 is the only lipid receptor among several tested to be present in the Virus-Containing Compartment (VCCs) in primary human macrophages infected by HIV-1. Silencing of CD36 expression in HIV-1-infected primary macrophages efficiently reduces the release of HIV-1 particles and the amount of intracellular Gag. Time-lapse microscopy of HIV-1-infected macrophages expressing Gag and CD36 fluorescent fusion proteins reveals that Gag can reach preexisting CD36+ compartments. Antibodies specific for CD36 are transported by infected macrophages into VCCs at 37° C. but not at 4° C., whereas SRB1-specific antibodies are also internalized but in structures distinct from VCCs. Importantly, exposure of HIV-1-infected macrophages to antibodies specific for CD36 for 24 h, or even less, led to strong reductions of the released viral particles that accumulate intracellularly instead.

The inventors have further shown that the anti-CD36 mAb exposure induces the retention of virions within the VCCs of infected cells, increasing the volume of the VCCS and thus inhibiting the particle release to the extracellular medium.

Since CD36 expression is restricted to a few tissues, these results open the door for new cell-specific therapies to target VCCs and their viral particles, and thus viral reservoirs constituted by HIV-infected macrophages.

### DEFINITIONS

The terms “CD36” and “CD36 receptor” are used herein interchangeably. In particular, it refers to human CD36. CD36 (Uniprot/Swiss-Prot accession number: P16671) is a multifunctional receptor that is expressed in various types of cells and tissues including brain microglia and astrocytes as well as monocytes/macrophages and adipocytes. CD36 consists of 472 amino acids with two cytoplasmic tails, two transmembrane domains and a large extracellular domain (from residue 30 to residue 439 of the mature protein Uniprot P16671). CD36 recognizes many ligands including oxidized or modified low density lipoprotein (oxLDL, mLDL), long-chain fatty acids (LCFA), lipid and lipoprotein components of bacterial cell walls, thrombospondin (TSP)-1 and -2, fibrillar [beta]-amyloid ([iota][Alpha][beta]), and dying cells (for detailed review see Febbraio, M., et ah, J Clin Invest 108:785-791 (2001); Febbraio, M., et al., Int J Biochem Cell Biol 39:2012-2030 (2007)). Ligand recognition by CD36 initiates a signaling cascade that, on phagocytic cells such as macrophages, leads to phagocytosis of the ligand (such as lipids and fatty acids) and any other material and the ligand may be bound to, such as cellular components, bacteria, etc. Internalization of oxLDL by CD36-expressing macrophages can lead to the formation of lipid-rich “foam cells” and atherosclerotic plaque, proinflammatory reactions, cytokine release, and/or production of reactive oxygen species, while internalization of [beta]-amyloid peptide by CD36-expressing microglial cells may contribute to Alzheimer's disease (Silverstein R L et al, Sci Signal 2:re3 (2010)). As a consequence of its broad expression and ability to recognize many ligands, CD36 participates in the regulation of many processes including inflammation, angiogenesis, native immunity, clearance of foreign and native substances from the body, and lipid metabolism. It is now well established that, despite having very short intracytoplasmic domains, CD36 can serve as a signaling molecule. Antibodies to platelet CD36 were shown to coprecipitate the nonreceptor protein tyrosine kinases Fyn, Lyn, and Yes. (Huang M M, et al. Proc Natl Acad Sci USA. 1991; 88: 7844-7848)

Studies in other cellular systems have linked the signaling function of CD36 to recruitment/activation of src family kinases and activation of specific mitogen-activated protein (MAP) kinases. For example, on microvascular endothelial cells, thrombospondin-1 induces a CD36-dependent antiangiogenic, proapoptotic signal via activation of Fyn, caspase-3, and p38 MAP kinase. (Jimenez B, et al. Nat Med. 2000; 6: 41-48). Moreover, OxLDL induces phosphorylation of p38 and ERK in platelets.

On macrophages, exposure to oxLDL leads to recruitment of Lyn and activation of c-Jun N-terminal kinase (JNK)2 in a CD36-dependent manner. Increased CD36 expression/function is associated with the pathology of atherosclerosis, stroke, and neurodegenerative diseases (Febbraio, M., et al., J Clin Invest 108:785-791(2001)) but CD36 has never been described as an alternative in HIV treatments targeting host factors, especially host factors specific of viral reservoirs such as macrophages, in order to avoid immune viral escape.

As used herein, the term “subject” denotes a human being.

In the context of the invention, the term “treating” is used herein to characterize a therapeutic method or process that is aimed at (1) slowing down or stopping the progression, aggravation, or deterioration of the symptoms of the disease state or condition to which such term applies; (2) alleviating or bringing about ameliorations of the symptoms of the disease state or condition to which such term applies; and/or (3) reversing or curing the disease state or condition to which such term applies.

As used herein the term “polypeptide” refers to any chain of amino acids linked by peptide bonds, regardless of length or post-translational modification. Polypeptides include natural proteins, synthetic or recombinant polypeptides and peptides (i.e. polypeptides of less than 50 amino acids) as well as hybrid, post-translationally modified polypeptides, and peptidomimetic.

As used herein, the term “amino acid” refers to the 20 standard alpha-amino acids as well as naturally occurring and synthetic derivatives. A polypeptide may contain L or D amino acids or a combination thereof

As used herein the term “peptidomimetic” refers to peptide-like structures which have non-amino acid structures substituted but which mimic the chemical structure of a peptide and retain the functional properties of the peptide. Peptidomimetics may be designed in order to increase peptide stability, bioavailability, solubility, etc.

As used here, the term “aptamer” means a molecule of nucleic acid or a peptide able to bind CD36. It refers to a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., Science, 1990, 249(4968):505-10. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., Clin. Chem., 1999, 45(9):1628-50. Peptide aptamers consist of a conformationally constrained antibody variable region displayed by a platform protein, such as E. coli Thioredoxin A that are selected from combinatorial libraries by two hybrid methods (Colas et al., Nature, 1996, 380, 548-50).

As used herein, the terms “antibody” and “immunoglobulin” have the same meaning and are used indifferently in the present invention. The term “antibody” refers to immunoglobulin molecules and immunologically active portions of immunoglobulin molecules, i.e., molecules that contain an antigen-binding site that immunospecifically binds an antigen. As such, the term antibody encompasses not only whole antibody molecules, but also antigen-binding antibody fragments as well as variants (including derivatives) of antibodies and antibody fragments. In particular, the antibody according to the invention may correspond to a polyclonal antibody, a monoclonal antibody (e.g. a chimeric, humanized or human antibody), a fragment of a polyclonal or monoclonal antibody or a diabody.

In natural antibodies, two heavy chains are linked to each other by disulfide bonds and each heavy chain is linked to a light chain by a disulfide bond. Each chain contains distinct sequence domains. The light chain includes two domains, a variable domain (V_{L}) and a constant domain (C_{L}). The heavy chain includes four domains, a variable domain (V_{H}) and three constant domains (C_{H}1, C_{H}2 and C_{H}3, collectively referred to as C_{H}). The variable regions of both light (V_{L}) and heavy (V_{H}) chains determine binding recognition and specificity to the antigen. Are also contemplated camelid antibodies, such as heavy-chain antibodies, and fragments and derivatives thereof such (VHH)₂ fragments, nanobodies and sdAb.

The specificity of the antibody resides in the structural complementarity between the antibody combining site and the antigenic determinant. Antibody combining sites are made up of residues that are primarily from the hypervariable or complementarity determining regions (CDRs). They refer to amino acid sequences which, together, define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. Therefore, an antigen-binding site includes six CDRs, comprising the CDR set from each of a heavy and a light chain V region.

Framework regions (FRs) refer to amino acid sequences interposed between CDRs, i.e. to those portions of immunoglobulin light and heavy chain variable regions that are relatively conserved among different immunoglobulins in a single species, as defined by Kabat et al., 1991 (Kabat et al., 1991, Sequences of Proteins Of Immunological Interest, National Institute of Health, Bethesda, Md.). As used herein, a “human framework region” is a framework region that is substantially identical (about 85%, or more, in particular, 90%, 95% or 100%) to the framework region of naturally occurring human antibody.

The term “monoclonal antibody” or “mAb” as used herein refers to an antibody molecule of a single amino acid composition, that is directed against a specific antigen and which may be produced by a single clone of B cells or hybridoma, or by recombinant methods. A “humanized antibody” is a chimeric, genetically engineered, antibody in which the CDRs from an antibody, e.g. a mouse antibody, (“donor antibody”) are grafted onto a human antibody (“acceptor antibody”). Thus, a humanized antibody is an antibody having CDRs from a donor antibody and variable region framework and constant regions from a human antibody. The use of antibody components derived from humanized monoclonal antibodies obviates potential problems associated with the immunogenicity of murine constant regions. Antibodies according to the invention may be produced by any technique known in the art, such as, without limitation, any chemical, biological, genetic or enzymatic technique, either alone or in combination. The antibodies of this invention can be obtained by producing and culturing hybridomas.

The term “Heavy-chain antibodies” or “HCAbs” refer to immunoglobulins which are devoid of light chains and consist in two heavy chains. These antibodies do not rely upon the association of heavy and light chain variable domains for the formation of the antigen-binding site but instead the variable domain of the heavy polypeptide chains alone naturally form the complete antigen binding site. Each heavy chain comprises a constant region (CH) and a variable domain which enables the binding to a specific antigen, epitope or ligand. As used herein, HCAbs encompass heavy chain antibodies of the camelid-type in which each heavy chain comprises a variable domain called VHH and two constant domains (CH2 and CH3). Such heavy-chain antibodies directed against a specific antigen can be obtained from immunized camelids. Camelids encompass dromedary, camel lama and alpaca. Camelid HCAbs have been described by Hamers-Casterman et al., 1993. Other examples of HCAb are immunoglobulin-like structures (Ig-NAR) from cartilaginous fishes. Heavy-chain antibodies can be humanized using well-known methods.

“Antibody fragments” comprise a portion of an intact antibody, preferably the antigen binding or variable region of the intact antibody. Examples of antibody fragments include Fv, Fab, F(ab′)2, Fab′, Fd, dAb, dsFv, scFv, di-scFvs, sc(Fv)₂, CDRs, VHH, (VHH)₂, diabodies and multi-specific antibodies formed from antibodies fragments.

The term “Fab” denotes an antibody monovalent fragment having a molecular weight of about 50,000 and antigen binding activity, and consisting of the V_{L}, V_{H}, C_{L} and C_{H}1 domains which can be obtained by cutting a disulfide bond of the hinge region of the F(ab′)2 fragment. The Fv fragment is the N-terminal part of the Fab fragment and consists of the variable portions of one light chain and one heavy chain.

The term “F(ab′)2” refers to an antibody bivalent fragment having a molecular weight of about 100,000 and antigen binding activity, which comprises two Fab fragments linked by a disulfide bridge at the hinge region.

The term “Fd” refers to an antibody fragment consisting of the V_{H} and C_{H}1 domains.

The term “dAb” (Ward et al., 1989 Nature 341:544-546) refers to a single variable domain antibody, i.e. an antibody fragment which consists of a V_{H} or V_{L} domain.

A single chain Fv (“scFv”) polypeptide is a covalently linked V_{H}::V_{L} heterodimer which is usually expressed from a gene fusion including V_{H} and V_{L} encoding genes linked by a peptide-encoding linker. “dsFv” is a V_{H}::V_{L} heterodimer stabilised by a disulfide bond. Divalent and multivalent antibody fragments can form either spontaneously by association of monovalent scFvs such as di-scFvs, or can be generated by coupling monovalent scFvs by a peptide linker, such as divalent sc(Fv)_{2.}

The term “diabodies” refers to small antibody fragments with two antigen-binding sites, which fragments comprise a V_{H} domain connected to a V_{L} domain in the same polypeptide chain (V_{H}-V_{L}). By using a linker that is too short to allow pairing between the two domains on the same chain, the domains are forced to pair with the complementarity domains of another chain and create two antigen-binding sites. The diabody may be mono- or bi-specific.

The term “VHH” refers to an antibody fragment consisting of the V_{H} domain of camelid heavy-chain antibody. VHH fragments can be produced through recombinant DNA technology in a number of microbial hosts (bacterial, yeast, mould), as described in WO 94/29457. Alternatively, binding domains can be obtained by modification of the V_{H} fragments of classical antibodies by a procedure termed “camelisation”, described by Davies et al, 1995. Dimers of VHH fragments, i.e. (VHH)₂, can be generated by fusing two sequences encoding VHH fragments, end to end, e.g. by PCR. Preferably, the (VHH) fragment is monospecific.

Rodent monoclonal antibodies to specific antigens may be obtained by methods known to those skilled in the art. (See, e.g., Kohler and Milstein, Nature 256: 495 (1975), and Coligan et al. (eds.), CURRENT PROTOCOLS IN IMMUNOLOGY, VOL. 1, pages 2.5.1-2.6.7 (John Wiley & Sons 1991)).

General techniques for cloning murine immunoglobulin variable domains have been disclosed, for example, by the publication of Orlandi et al., Proc. Nat'l Acad. Sci. USA 86: 3833 (1989). Techniques for constructing chimeric antibodies are well known to those of skill in the art. As an example, Leung et al., Hybridoma 13:469 (1994), disclose how they produced an LL2 chimera by combining DNA sequences encoding the Vk and VH domains of LL2 monoclonal antibody, an anti-CD22 antibody, with respective human and IgG1 constant region domains. This publication also provides the nucleotide sequences of the LL2 light and heavy chain variable regions, Vk and VH, respectively. Techniques for producing humanized antibodies are disclosed, for example, by Jones et al., Nature 321: 522 (1986), Riechmann et al., Nature 332: 323 (1988), Verhoeyen et al., Science 239: 1534 (1988), Carter et al., Proc. Nat'l Acad. Sci. USA 89: 4285 (1992), Sandhu, Crit. Rev. Biotech. 12: 437 (1992), and Singer et al., J. Immun. 150: 2844 (1993).

In one alternative, the phage display technique may be used to generate human antibodies (e.g., Dantas-Barbosa et al., 2005, Genet. Mol. Res. 4:126-40, incorporated herein by reference). Human antibodies may be generated from normal humans or from humans that exhibit a particular disease state, such as cancer (Dantas-Barbosa et al., 2005). The advantage to constructing human antibodies from a diseased individual is that the circulating antibody repertoire may be biased towards antibodies against disease-associated antigens. In one non-limiting example of this methodology, Dantas-Barbosa et al. (2005) constructed a phage display library of human Fab antibody fragments from osteosarcoma patients. Generally, total RNA was obtained from circulating blood lymphocytes (Id.) Recombinant Fab were cloned from the [mu], [gamma] and [kappa] chain antibody repertoires and inserted into a phage display library (Id.) RNAs were converted to cDNAs and used to make Fab cDNA libraries using specific primers against the heavy and light chain immunoglobulin sequences (Marks et al., 1991, J. Mol. Biol. 222:581-97). Library construction was performed according to Andris-Widhopf et al. (2000, In: Phage Display Laboratory Manual, Barbas et al. (eds), 1<st> edition, Cold Spring Harbor Laboratory Press, Cold Spring Harbor, N.Y. pp. 9.1 to 9.22, incorporated herein by reference). The final Fab fragments were digested with restriction endonucleases and inserted into the bacteriophage genome to make the phage display library. Such libraries may be screened by standard phage display methods. The skilled artisan will realize that this technique is exemplary only and any known method for making and screening human antibodies or antibody fragments by phage display may be utilized.

In another alternative, transgenic animals that have been genetically engineered to produce human antibodies may be used to generate antibodies against essentially any immunogenic target, using standard immunization protocols as discussed above. Methods for obtaining human antibodies from transgenic mice are described by Green et al., Nature Genet. 7:13 (1994), Lonberg et al., Nature 368:856 (1994), and Taylor et al., Int. Immun. 6:579 (1994). A non-limiting example of such a system is the XenoMouse® (e.g., Green et al., 1999, J. Immunol. Methods 231:11-23, incorporated herein by reference) from Abgenix (Fremont, Calif.). In the XenoMouse® and similar animals, the mouse antibody genes have been inactivated and replaced by functional human antibody genes, while the remainder of the mouse immune system remains intact.

Antibody fragments which recognize specific epitopes can be generated by known techniques. The antibody fragments are antigen binding portions of an antibody, such as F(ab)2, Fab′, Fab, Fv, scFv and the like. Other antibody fragments include, but are not limited to: the F(ab′)2 fragments which can be produced by pepsin digestion of the antibody molecule and the Fab′ fragments, which can be generated by reducing disulfide bridges of the F(ab′)2 fragments. Alternatively, Fab′ expression libraries can be constructed (Huse et al., 1989, Science, 246:1274-1281) to allow rapid and easy identification of monoclonal Fab′ fragments with the desired specificity.

In a first aspect, the present invention concerns a composition comprising an antibody that recognizes and binds CD36, or an antigen-binding fragment or derivative thereof, a CD36 antagonist, or a conjugate comprising at least one CD36 targeting agent associated to at least one anti-HIV effector moiety, and a pharmaceutically acceptable excipient. More particularly, the present invention concerns said composition for use in the treatment of HIV infection.

### CD36 Antagonists

The inventors showed that silencing of CD36 using siRNA or exposure of infected macrophages to an anti-CD36 monoclonal antibody leads to the intracellular retention of viral particles and thus inhibits their release in the intracellular medium.

Thus, in a first embodiment, the composition comprises a CD36 antagonist. The present invention thus provides new therapeutic agents for treating HIV infection, namely CD36 antagonists.

As used herein the term “antagonist”, or inhibitor as used interchangeably, refers to an agent (i.e. a molecule) which act by blocking or reducing CD36 receptor functional activation. This may for example be achieved by reducing, affecting or preventing expression or activity of CD36 receptor per se, preferably through direct binding to a CD36 receptor molecule or gene. Alternatively, this may be achieved by interfering with a protein from one of the pathways activated by CD36 as described above, such as c-Jun, Lyn and Erk. Both options ultimately result in blocking or reducing signal transduction, hence in blocking or reducing CD36 receptor functional activity. Preferred within the context of the invention are those CD36 antagonists acting through direct interaction with the CD36 receptor or CD36 encoding nucleic acid to block its activity or expression.

The antagonists according to the invention are capable of inhibiting or eliminating the functional activation of the CD36 receptor in vivo and/or in vitro. The antagonist may inhibit the functional activation of the CD36 receptor by at least about 30%, preferably by at least about 50%, more preferably by at least 70%, or 80%, even more preferably by at least about 90%, 95%, 97%, 98% or 99%.

Functional activation of the CD36 receptor may be readily assessed by the one skilled in the art according to known methods. Indeed, since activated CD36 receptor is internalized with its ligand, i.e. LDLox, functional activation of the CD36 receptor may for example be assessed by measuring its internalization and its transport into internal VCC. For instance, analysis of ligand-induced internalization of the CD36 receptor can be performed as described in examples. Quantification of the internalization of CD36 receptor can also be performed by using a lentivector encoding CD36-GFP as described in examples. For instance, analysis of ligand-induced internalization of the CD36 receptor can be performed using a cytometry-based assay as described in Ning Li et al.

Alternatively, CD36 mediates signaling by activating, for example, the Erk or Jun pathway phosphorylation. This inhibition of phosphorylation in cells can be readily detected by immunocytochemistry, immunohistochemistry and/or flow cytometry using antibodies which specifically recognize this modification. For instance, phosphorylation can be detected by immunocytochemistry, immunohistochemistry and/or flow cytometry using monoclonal or polyclonal antibodies directed against phosphorylated proteins of this specific pathway.

The antagonists according to the present invention include those which specifically bind either to the CD36 polypeptide, or to its ligand, or to a protein of the CD36 pathway thereby reducing or blocking signal transduction. Antagonists of this type include antibodies or aptamers which bind to the receptors, antibodies or aptamers which bind to the ligands, soluble ligands which bind to the receptor, soluble receptors which bind to their ligands, fusion polypeptides, peptides, small molecules, chemicals and peptidomimetics. Preferred are antagonists which bind to the CD36 receptor, preferably via direct interaction.

According to a preferred embodiment, the CD36 antagonist is an antibody which specifically recognizes and binds to the CD36 receptor or a fragment thereof, or to its ligand LDLox or to a fragment thereof. In an even preferred embodiment, the antagonist is an antibody which specifically recognizes and binds to the CD36 receptor or a fragment thereof. Optionally, the antibody specifically recognizes and binds to a fragment of the extracellular domain of CD36. Even more preferably, the inhibitor is a function-blocking monoclonal antibody against CD36.

The term “function-blocking” refers to an inhibitor that blocks CD36 functions by inhibiting the release of HIV particles from CD36 expressing cells.

Preferably, the antagonist is a neutralizing antibody. In the context of the present invention, the term “neutralizing antibody” refers to an antibody which is capable of binding to the CD36 receptor and blocking CD36 binding to its ligand, to an antibody which is capable of binding to the LDLox ligand and preventing LDLOx binding of its CD36 receptor. Are also contemplated antibodies that specifically recognize and bind to the CD36 receptor and reduce or block signal transduction by the CD36 receptor, even without interfering with the CD36 binding to its ligands.

Regarding the antibodies used as antagonists of CD36 receptor, they are preferably elicited against the extracellular region of CD36, more preferably against the immunoglobulin-like domain, even more preferably against the domain involved into the interaction with CD36 ligand, such as the domain encoded by amino acids 155-183 (Puente Navazo et al, 1996, Arterioscler Thromb Vasc Biol, 16(8): 1033-9). Examples of such CD36 antibodies include clone FA6-152 or IVC-7 from Immunotech (Beckman Coulter, Fullerton, Calif.). CD36 antagonist antibodies can also be obtained using competition assays with known CD36 antibodies directed against the domain of CD36 involved into the interaction with CD36 ligand such as FA6-152 or IVC-7 mAbs.

Preferably, the antibody used as CD36 antagonist is a full-length antibody. However, antigen-binding fragments or derivatives can also be used. Indeed, the inventors demonstrated that Fc receptors are not involved in the antibody-mediated effect on the inhibition of HIV particles release.

The antibody is preferably a monoclonal antibody. In particular, the antibody may be a humanized, chimeric or human antibody, preferably a human antibody.

Depending on the type of constant domain in the heavy chains, antibodies are assigned to one of five major classes: IgA, IgD, IgE, IgG, and IgM. Preferably, the antibody is of the IgG, IgE or IgD type, more preferably IgG type.

The antibody may also be a camelid heavy-chain antibody, and in particular humanized camelid heavy-chain antibody.

In a preferred embodiment, the antibody, or antigen-binding fragment or derivative thereof, is bivalent. In particular, the antigen-binding fragment or derivative retains the antigen specificity of the full-length antibody and is bivalent. The antigen-binding fragment or derivative may be selected, for example, from the group consisting of F(ab′)2, di-scFvs, sc(Fv)₂ fragments, (VHH)₂ fragments and diabodies.

In another embodiment, the CD36 antagonist is a small molecule.

The term “small molecule” refers to a molecule of less than about 1500 Daltons, 1000 Daltons, 800 Daltons, or even less than about 500 Daltons, in particular organic or inorganic compounds. Structural design in chemistry should help to find such a molecule. The molecule may have been identified by a screening method disclosed in the present invention. In the context of the present invention, the term “small molecule” refers to small organic compounds, such as heterocycles, peptides, saccharides, steroids, and the like. The compounds may be modified to enhance efficacy, stability, pharmaceutical compatibility, and the like.

Candidate modulator compounds from libraries of synthetic or natural compounds can be screened by methods known in the art (see Chinese patent number ZL200510072269.1, Publication No: CN1868546, and Chinese patent application number 200910147634.9, each hereby incorporated in its entirety by reference).

Synthetic compound libraries are commercially available from a number of companies including Maybridge Chemical Co. (Trevillet, Cornwall, UK), Comgenex (Princeton, N. J.), Brandon Associates (Merrimack, N.H.), and Microsource (New Milford, Conn.). Combinatorial libraries are available or can be prepared according to known synthetic techniques.

Alternatively, libraries of natural compounds in the form of bacterial, fungal, plant and animal extracts are available from e.g., Pan Laboratories (Bothell, Wash.) and MycoSearch (NC), or are readily producible by methods well known in the art.

Additionally, natural and synthetically produced libraries and compounds can be further modified through conventional chemical and biochemical techniques.

The use of high throughput screening has allowed the identification of compounds that potently antagonize CD36 in vitro. Following isolation of CD36 antagonists by in vitro testing, experiments can be conducted to test the efficacy in vivo.

For example, a lead compound, salvianolic acid B (SAB; CAS Registry Number 115939-25-8), has been identified as a CD36 antagonist in a high throughput screen. SAB metabolizes into rosmarinic acid (RA: CAS Registry number 20283-92-5) and sodium danshensu (DSS; CAS Registry number 67920-52-9).

In certain embodiments, the small molecule antagonist of CD36 activity can be selected from SAB, its metabolites RA and DSS (as described above), and derivatives thereof.

SAB or its metabolites can be derivatized, for example, as esters, ethers, oximes, hydrazones, hydroxyamines, carbamate esters, alkoxyesters, or carbonate esters in 1-3 enzymatic steps, or PEG derivatives.

Other small molecule antagonists of CD36 have been identified through high-throughput screening, for example the following two structures, 3-cinnamoyl indole, and 13 pentyl berberine (Xu, Y et al., Anal Biochem 400(2): 207-212 (2010)): S-cirinarnoy Indole 13-enty.

Another example of a small molecule CD36 antagonist is hexarelin, a member of the hexapeptide growth hormone-releasing peptides (GHRPs) family that possesses growth hormone releasing activity and binds to CD36 receptors (Demers A et al., Biochem J. 382(Pt 2):417-24(2004)). Hexarelin blocks CD36-mediated uptake of oxLDLs through binding site competition, since the binding domain of hexarelin on CD36 overlaps that of oxLDLs (Demers A et al, Biochem J. 382(Pt 2):417-24(2004)).

Other antagonists against scavenger receptors include synthetically engineered nanoblockers. After self-assembling into 15-20 nm ligand units, these nanoparticles block the major scavenger receptors from oxLDLs uptake and thus prevent formation of foam cells (Chnari E, et al., Biomacromolecules. 7(6): 1796-805(2006)).

Statins or antioxidants can modulate CD36 expression or its downstream effects. Some statins and the antioxidant α-tocopherol can reduce expression of CD36 and the uptake of oxLDLs into macrophages (Venugopal S K, et al., Atherosclerosis. 175(2):213-20 (2004), Ricciarelli R, et al., Circulation 102: 82-87 (2000), Fuhrman B, et al., Atherosclerosis 164(1): 179-85 (2002)). Another class of antioxidants is SS peptides. The SS peptides SS31 has been shown to attenuate ischemia-induced GSH depletion in the cortex and reduced infarct size. This attenuation has been observed to be abrogated in CD36 knock-out mice, indicating that the protective effect of SS31 occurs via down regulating CD36-mediated pathways (Cho S, et al., J Biol Chem. 282(7):4634-42 (2007)).

Other small molecule for CD36 receptor are well known in the art and include e.g. Sulfo-N-succinimidyl oleate (SSO), Ursolic acid, AP5258 (Arteria).

Thus, in a particular embodiment, the CD36 antagonist is a small molecule selected from the group consisting of salvianolic acid B, rosmarinic acid, sodium danshensu, 3-cinnamoyl indole, 13 pentyl berberine, hexarelin, nanoblockers, statins or antioxidants such as α-tocopherol and SS peptides, Sulfo-N-succinimidyl oleate and Ursolic acid, and any combination thereof. Preferably, the small molecule is selected from the group consisting of Sulfo-N-succinimidyl oleate (SSO), Ursolic acid, AP5258 (Arteria), and any combination thereof, more preferably selected from Sulfo-N-succinimidyl oleate and Ursolic acid, and combination thereof.

Antagonists according to the present invention also include molecules which reduce or prevent the expression of CD36, such as a nucleic acid molecule interfering specifically with CD36 expression. Thus, in a further embodiment, the CD36 antagonist is a nucleic acid molecule interfering specifically with CD36 expression. Such nucleic acid is, for example, an antisense oligonucleotide comprising a single-stranded polynucleotide sequence (either RNA or DNA) capable of binding to target mRNA (sense) or DNA (antisense) sequences, an interfering RNA (iRNA), or a ribozyme. Said nucleic acid can have a sequence from 15 to 50 nucleotides, preferably from 15 to 30 nucleotides.

The decrease or the inhibition of CD36 expression can be evaluated by any means known to those skilled in the art including, but not limited to, assessing the level of the CD36 protein of interest using Western Blot analysis, using any suitable anti-CD36 antibody, and/or assessing the level of mRNA for CD36 using any available technique such as qPCR for example. The expression level of a gene can be determined by measuring the quantity of mRNA or the protein encoded by this gene using any technique known by the skilled person. The quantity of a specific mRNA may be measured, for instance, by quantitative or semi-quantitative RT-PCR, by real-time quantitative or semi-quantitative RT-PCR or by transcriptomic approaches. The quantity of a protein may be measured, for instance, by immunohistochemistry or semi-quantitative western-blot. Expression levels obtained from samples may be normalized by using expression levels of proteins which are known to have stable expression such as RPLPO (acidic ribosomal phosphoprotein PO) (de Cremoux et al., 2004), TBP (TATA box binding protein), GAPDH (glyceraldehyde 3-phosphate dehydrogenase) or β-actin.

Alternatively, CD36 can be detected by immunocytochemistry, immunohistochemistry and/or flow cytometry using monoclonal or polyclonal antibodies directed against CD36. By a “decrease” in expression is meant, for example, a decrease of at least 30%, preferably at least 50%, more preferably at least 70%, or 80%, even more preferably at least 90%, 95%, 97, 98 or 99% of the gene expression product.

Ribozymes can also function as inhibitors of the expression of the CD36 receptor or of its ligand for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of CD36 receptor is thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Ribozyme molecules can also be used to decrease levels of functional CD36. Ribozymes are catalytic RNA molecules with ribonuclease activity which are capable of cleaving a single-stranded nucleic acid, such as an mRNA, to which they have a complementary region. Thus, ribozymes can be used to catalytically cleave mRNA transcripts to thereby inhibit translation of the protein encoded by the mRNA. Ribozyme molecules specific for functional CD36 can be designed, produced, and administered by methods commonly known to the art (see e.g., Fanning and Symonds, 2006, reviewing therapeutic use of hammerhead ribozymes and small hairpin RNA) using, for example, CD36 shRNA Plasmid (h): sc-29995-SH, CD36 siRNA (h): sc-29995, CD36 shRNA (h) Lentiviral Particles: sc-29995-V(Santa Cruz biotechnology).

Antisense or sense oligonucleotides comprise fragments of the targeted polynucleotide sequence encoding CD36 receptor or its ligand. Such a fragment generally comprises at least about 14 nucleotides, typically from about 14 to about 30 nucleotides. The ability to derive an antisense or a sense oligonucleotide, based upon a nucleic acid sequence encoding a given protein is described in, for example, Stein and Cohen (Cancer Res., 1988, 48:2659), and van der Krol et al. (BioTechniques, 1988, 6:958).

Binding of antisense or sense oligonucleotides to target nucleic acid sequences results in the formation of duplexes that block or inhibit protein expression by one of several means, including enhanced degradation of the mRNA by RNAse H, inhibition of splicing, premature termination of transcription or translation, or by other means. The antisense oligonucleotides thus may be used to block expression of proteins. Antisense or sense oligonucleotides further comprise oligonucleotides having modified sugar-phosphodiester backbones (or other sugar linkages, such as those described in WO 91/06629) and wherein such sugar linkages are resistant to endogenous nucleases. Such oligonucleotides with resistant sugar linkages are stable in vivo (i.e., capable of resisting enzymatic degradation) but retain sequence specificity to be able to bind to target nucleotide sequences.

Other examples of sense or antisense oligonucleotides include those oligonucleotides which are covalently linked to moieties that increases affinity of the oligonucleotide for a target nucleic acid sequence, such as poly-(L)-lysine. Further still, intercalating agents, such as ellipticine, and alkylating agents or metal complexes may be attached to sense or antisense oligonucleotides to modify binding specificities of the antisense or sense oligonucleotide for the target nucleotide sequence.

Antisense or sense oligonucleotides may be introduced into a cell containing the target nucleic acid by any gene transfer method, including, for example, electroporation, microinjection, transduction, cell fusion, DEAE dextran, calcium phosphate precipitation, use of a gene gun, or lipofection, or by using gene transfer vectors such as Epstein-Barr virus or adenovirus.

An antisense nucleic acid for use in the method of the invention can be constructed using chemical synthesis and enzymatic ligation reactions using procedures known in the art. Particularly, antisense RNA can be chemically synthesized, produced by in vitro transcription from linear (e.g. PCR products) or circular templates (e.g., viral or non-viral vectors), or produced by in vivo transcription from viral or non-viral vectors.

Antisense nucleic acid may be modified to have enhanced stability, nuclease resistance, target specificity and improved pharmacological properties. For example, antisense nucleic acid may include modified nucleotides designed to increase the physical stability of the duplex formed between the antisense and sense nucleic acids, e.g., phosphorothioate derivatives and acridine substituted nucleotides.

Sense or antisense oligonucleotides also may be introduced into a cell containing the target nucleic acid by formation of a conjugate with a ligand-binding molecule. Suitable ligand binding molecules include, but are not limited to, cell surface receptors, growth factors, other cytokines, or other ligands that bind to cell surface receptors. Preferably, conjugation of the ligand-binding molecule does not substantially interfere with the ability of the ligand-binding molecule to bind to its corresponding molecule or receptor, or block entry of the sense or antisense oligonucleotide or its conjugated version into the cell.

Additional methods for preventing expression of the CD36 receptor or its ligand LDLOx include RNA interference (RNAi). RNAi is carried out through the use of an interfering RNA (iRNA) which is capable of down-regulating the expression of the targeted protein. As used herein, the term “iRNA” encompasses small interfering RNA (siRNA), double-stranded RNA (dsRNA), single-stranded RNA (ssRNA), micro-RNA (miRNA) and short hairpin RNA (shRNA) molecules, specific for pre-mRNA or mRNA of said receptor or said ligand. Short hairpin RNAs (shRNA) will be cleaved by the cellular machinery into siRNA specific for pre-mRNA or mRNA of said receptors or said ligands (Paddison et al., Genes & Development, 16: 948-958, 2002).

RNA interference designates a phenomenon by which dsRNA specifically suppresses expression of a target gene at post-translational level.

In normal conditions, RNA interference is initiated by double-stranded RNA molecules (dsRNA) of several thousands of base pair length. In vivo, dsRNA introduced into a cell is cleaved into a mixture of short dsRNA molecules called siRNA. The enzyme that catalyzes the cleavage, Dicer, is an endo-RNase that contains RNase III domains (Bernstein et al., Nature, 409: 363-366, 2001). In mammalian cells, the siRNAs produced by Dicer are 21-23 bp in length, with a 19 or 20 nucleotides duplex sequence, two-nucleotide 3′ overhangs and 5′-triphosphate extremities (Elbashir et al., Nature, 411: 494-498, 2001; Elbashir et al., Genes. Dev. 15(2): 188-200, 2001; Zamore et al., Cell, 101(1): 25-33, 2000). A number of patents and patent applications have described, in general terms, the use of siRNA molecules to inhibit gene expression, for example, WO 99/32619, US 20040053876, US 20040102408 and WO 2004/007718.

siRNA are usually designed against a region 50-100 nucleotides downstream the translation initiator codon, whereas 5′UTR (untranslated region) and 3′UTR are usually avoided. The chosen siRNA target sequence should be subjected to a BLAST search against EST database to ensure that the only desired gene is targeted. Various products are commercially available to aid in the preparation and use of siRNA. In a preferred embodiment, the RNAi molecule is a siRNA of at least about 15-50 nucleotides in length, preferably about 20-30 base nucleotides.

RNAi can comprise naturally occurring RNA, synthetic RNA, or recombinantly produced RNA, as well as altered RNA that differs from naturally-occurring RNA by the addition, deletion, substitution and/or alteration of one or more nucleotides. Such alterations can include addition of non-nucleotide material, such as to the end of the molecule or to one or more internal nucleotides of the RNAi, including modifications that make the RNAi resistant to nuclease digestion.

RNAi may be administered in free (naked) form or by the use of delivery systems that enhance stability and/or targeting, e.g., liposomes, or incorporated into other vehicles, such as hydrogels, cyclodextrins, biodegradable nanocapsules, bioadhesive microspheres, or proteinaceous vectors (WO 00/53722), or in combination with a cationic peptide (US 2007275923). They may also be administered in the form of their precursors or encoding DNAs.

In a preferred embodiment, the nucleic acid molecule interfering specifically with CD36 expression is a RNAi, an antisens nucleic acid or a ribozyme, preferably a RNAi.

In a particular embodiment, the nucleic acid molecule interfering specifically with CD36 expression is a siRNA molecule comprising a sequence selected from the group consisting of the sequence of siCD36_{—}1 of SEQ ID No.1 (GAACCUAUUGAUGGAUUAATT), siCD36_{—}3 of SEQ ID No.2 (CCUUCACUAUCAGUUGGAATT), and siCD36_{—}4 of SEQ ID No.3 (GCAACAUUCAAGUUAAGCATT).

In a particular embodiment, RNAi are encapsulated within vesicles, preferably within liposomes.

The interfering RNA, the antisense nucleic acids and the ribozyme molecule used according to the invention can be administered in the form of DNA precursors or molecules coding for them.

Antibodies that Recognize and Bind CD36, or Antigen Binding Fragments Thereof or Derivatives Thereof

The inventors showed that the exposure of infected macrophages to an anti-CD36 antibody induces virions tethering at the site of viral assembly, i.e. within the VCCs. They further demonstrated that the anti-CD36 antibody effect on particle release is not related to the pathways induced upon CD36 binding to some of its endogenous ligands such as MOxLDL, trombospondin I or type-1 collagen. Moreover, this effect does not require the integrity of the CD36 cytoplasmic tail in charge of the signal transduction.

Thus, in a second embodiment, the composition comprises an antibody that recognizes and binds CD36, or an antigen binding fragment thereof or derivative thereof. Said antibody being or not a CD36 antagonist.

The antibody is directed against the extracellular loop of CD36 (residues 30 to 439 of the mature protein, Uniprot P16671). Such antibody can be obtained using any method known by the skilled person, in particular using the extracellular loop, or a portion thereof, as elicitor. In a particular embodiment, the antibody is directed against the region comprising residues 155 to 183. Examples of such CD36 antibodies include clone FA6-152 or IVC-7 from Immunotech (Beckman Coulter, Fullerton, Calif.).

Antibodies useful in the present invention can also be obtained using competition assay with known CD36 antibodies directed against the extracellular loop such as FA6-152 or IVC-7 mAbs.

The CD36 antibody may be a full-length antibody or an antigen-binding fragment or derivative thereof. In particular, the inventors demonstrated that Fc receptors are not involved in the antibody-mediated effect on the inhibition of HIV particles release. Fragments and derivatives without Fc region may thus be used in the present invention.

The antibody may be a polyclonal or monoclonal antibody, preferably a monoclonal.

The antibody may be a humanized, chimeric or human antibody, preferably a human antibody. The antibody may also be a camelid heavy-chain antibody, and in particular humanized camelid heavy-chain antibody.

Depending on the type of constant domain in the heavy chains, antibodies are assigned to one of five major classes: IgA, IgD, IgE, IgG, and IgM. Preferably, the antibody is of the IgG, IgE or IgD type, more preferably IgG type.

In a preferred embodiment, the antibody, or antigen-binding fragment or derivative thereof, is bivalent. In particular, the antigen-binding fragment or derivative retains the antigen monospecificity of the full-length antibody and is bivalent. The antigen-binding fragment or derivative may be selected, for example, from the group consisting of F(ab′)2, di-scFvs, sc(Fv)₂ fragments, (VHH)₂ fragments and diabodies.

### Conjugates Comprising at Least One CD36 Targeting Agent Associated to at Least One Anti-HIV Effector Moiety

HIV-1-infected macrophages are believed to represent an important viral reservoir due to their intracellular accumulation of Virus-Containing Compartments. The inventors have shown that CD36 inhibitors according to the invention are able, when they are introduced into infected macrophages, to induce internalisation of a CD36 into VCC. Consequently, molecules capable of binding CD36, e.g., inhibitors, can also be used as a vector, i.e. a mean to target to macrophages an effector molecule able to destroy HIV particles. In order to specifically target and eliminate these viral reservoirs, the CD36-specific targeting agent, e.g. a CD36-specific antagonist, could be coupled to a molecule effector.

Accordingly, the present invention is directed to new systems and strategies for improved delivery and administration of HIV therapy. More specifically, the present invention provides compositions for the selective targeting of HIV reservoir cells.

In one aspect, the present invention therefore provides conjugates, and methods for using these conjugates in the treatment of HIV infection.

In particular, the present invention encompasses the recognition that the conjugate (i) exhibits specificity for HIV reservoir cells expressing CD36, (ii) undergoes efficient cellular internalization, and (iii) is transported in a retrograde fashion from the plasma membrane to the VCC.

Accordingly, the present invention relates to the use of a CD36 targeting agent as selective carriers for HIV effector.

More specifically, in one aspect, the present invention provides a conjugate comprising at least one CD36 targeting agent associated to at least one anti-HIV effector moiety. In such conjugates, the CD36 targeting agent, which interacts with HIV reservoir cells that express CD36, helps to target and deliver the HIV effector to the VCC.

In some embodiments, the CD36 targeting agent and the anti-HIV effector are associated through a linker Such association may occur through recombinant linking (e.g., as a fusion protein) or through chemical conjugation according to methods know to those skilled in the art.

As can be appreciated by one skilled in the art, a conjugate of the present invention can be designed to comprise any number of CD36 targeting agents, of various or identical nature, and any number of anti-HIV effectors of various or identical nature, eventually associated through (a) linker(s).

Such conjugates may be administered alone or as part of an HAART combination, wherein the combination has synergy effects or additive effects in the treatment of HIV infection over the conjugate used alone, the HIV toxic agent used alone. Of particular interest is the use of a conjugate according to the invention in combination with another anti-viral treatment, in particular with an agent used in HAART as described herein, or in combination with HAART itself.

### CD36 Targeting Agent

CD36 targeting agents, as used herein, refer to any molecule able to bind to CD36 and be routed to VCC of HIV-infected macrophages. Such CD36 targeting agent may be selected among the CD36 antagonists according to the invention, but may also be non-CD36 antagonists as they do necessarily need to be able to reduce or inhibit CD36 functional activation. In particular, CD36 targeting agents include functional equivalents of CD36 antagonists, i.e. molecules with homologous sequences to CD36 antagonists but not necessarily having the ability to function as a CD36 antagonists. In a preferred embodiment, said CD36 targeting agent is an antibody recognizing, preferably specifically, and binding to CD36, or to a antigen-binding fragment or derivative thereof.

The term “functional equivalent” of CD36 antagonists, as used herein, includes antibodies with homologous sequences, chimeric antibodies, artificial antibodies and modified antibodies, for example, wherein each functional equivalent is defined by its ability to bind to the CD36 protein. Methods of producing functional equivalents are known to the person skilled in the art and are disclosed, for example, in WO 93/21319, EP 239,400; WO 89/09622; EP 338,745; and EP 332,424, which are incorporated in their respective entireties by reference.

In one embodiment, such CD36 targeting agent is able to be internalized into VCC of HIV-infected macrophages.

The scope of the present invention is not limited to antibodies and fragments comprising these sequences. Instead, all antibodies and fragments that specifically bind to CD36 and capable of being routed to VCC of HIV-infected macrophages and/or decreasing particle release (p24), fall within the scope of the present invention. Thus, antibodies and antibody fragments and derivatives may differ from antibody well known in the art or the humanized derivatives in the amino acid sequences of their scaffold, CDRs, light chain and heavy chain, and still fall within the scope of the present invention.

### Effector Molecules

It is known that CD4 binding induces conformational changes in the gp120 glycoprotein, some of which involve the exposure and/or formation of a binding site for specific chemokine receptors. These chemokine receptors, mainly CCR5 and CXCR4 for HIV-1, serve as obligate second receptors for virus entry. Consequently, CD36 could be used as a vector associated with a protein involved in the interaction with HIV envelope making the virus containing in the VCC no more infectious.

In conjugates provided by the present invention, the CD36 targeting agent is linked to an effector molecule. The effector molecule or molecules may affect the viral particles by any mean leading to an impaired infectivity of the viral particle. This includes molecule or molecules able to interact with the Env protein present at the surface of the viral particle or with other constituents of the viral membrane.

As described herein, the terms “anti-HIV effector moiety” or “effector molecule” refers to any anti-HIV agent or a molecule interacting with the virus, more preferably a molecule interacting with the virus envelope.

The effector molecule or molecules may be released from the conjugate by cleavage/dissociation in or at a target cell, tissue and/or organ.

Accordingly, the CD36 targeting agent, e.g. a CD36 antagonist, could be coupled to inhibitors of the HIV-1 Env or a protein analog, such as T20 or a CD4 binding domain to gp120, to inactivate viral envelope coupled to Fab or ScFv recognizing the CD36.

The effector molecule is preferably a HIV co-receptor, or a domain thereof able to bind to the Envelope.

The effector molecule may also be an anti-HIV agent, in particular selected from the group consisting of reverse transcriptase inhibitors, integrase inhibitors and protease inhibitors. The effector molecule may be a reverse transcriptase inhibitor, preferably selected from the group consisting of deoxythymidine, zidovudine, stavudine, didanosine, zalcitabine, abacavir, lamivudine, emtricitabine, entecavir, apricitabine, adefovir, efavirenz, nevirapine, delavirdine, etravirine, rilpivirine and tenofovir. The effector molecule may also be an integrase inhibitor, preferably selected from the group consisting of raltegravir, elvitegravir and MK-2048. The effector molecule may further be a protease inhibitor, preferably selected from the group consisting of lopinavir, indinavir, nelfinavir, amprenavir, ritonavir, saquinavir, atazanavir, fosamprenavir, tipranavir and darunavir.

### HIV Co-Receptor

The primary targets for HIV-I infection in vivo are CD4 T cells and cells of the monocyte/macrophage lineage (Klatzmann et al., 1984, Nature 312: 767-8; Dalgleish et al., 1984, Nature 312: 763-7). The initial, critical step of HIV infection is its cell entry through the fusion of the viral membrane with the membrane of either a T-cell or macrophage. Major advances have been made over the past decade in the understanding of the molecular machinery of HIV entry into these target cells. An initial step in the entry process is the interaction of the external HIV envelope glycoprotein, gp120, with T-cell CD4 receptor molecules. The functional HIV-I envelope complex is a trimeric structure comprising three gp120 surface glycoproteins, each noncovalently attached to one of three subunits of the gp41 transmembrane glycoproteins (Chan et al., 1997, Cell 89: 263-73; Wyatt et al., 1998, Science 280: 1884-8; Tan et al., 1997, Proc Natl Acad Sci USA 94: 12303-8). Recent crystal structures of gp120-CD4 with co-receptor surrogate antibody complexes have provided insights into the formation of protein-protein interactions in the process of viral entry (Kwong et al., 1998, Nature 393: 648-59; Huang et al., 2005, Structure 13: 755-68; Huang et al., 2005, Science 310: 1025-8). The binding of gp120 to CD4 receptor promotes a conformational rearrangement in the envelope gp120, that creates a new site for binding of another co-receptor, CCR5 or CXCR4 (Wu et al, 1996, Nature 384: 179-83; Dragic et al., 1996, Nature 381: 667-73). The interaction of virus envelope gp120-CD4 complex with co-receptor is believed to promote further conformational rearrangements in HIV-1 envelope that drive fusion of the viral and host cell membranes.

Accordingly, the present invention relates to a conjugate comprising at least one CD36 targeting agent, preferably an antibody which recognizes, preferably specifically, and binds to CD36, associated to at least one anti-HIV effector moiety, wherein said anti-HIV effector moiety is a HIV co-receptor. Such HIV co-receptor is involved in the fusion of the virus envelope and the plasma membrane of the host cells. The CD36 targeting moiety will allow transport of the conjugate from the plasma membrane to the VCC lumen. There, the conjugate may interact with viral particles through its co-receptor domain and thus promote the loss of the gp120 and the exposure of the gp41. The fusion peptide of the gp41 would thus be exposed and favor the fusion of the viral particles with neighboring membranes of cellular or viral origin.

Several HIV co-receptors on T cells and/or monocytes/macrophages for the virus have been identified and implicated in entry of the virus into cells. These chemokine co-receptors include CCR5, CXCR4, CD4, T20 and others. In addition, he small molecule NBD-556 mimics CD4 by binding to the gp120 exterior envelope glycoprotein (Navid Madani et al, Small-Molecule CD4 Mimics Interact with a Highly Conserved Pocket on HIV-1 gp120, 2008, Structure, 12: 16(11): 1689-701), a 27-amino acid CD4 mimic, CD4M33, presents optimal interactions with gp120 and binds to viral particles and diverse HIV-1 envelopes with CD4-like affinity (Loïc Martin et al, Rational design of a CD4 mimic that inhibits HIV-1 entry and exposes cryptic neutralization epitopes, 2008, Methods Mol Biol).

In addition, effective anti-HIV effector moiety may comprise functional equivalents or fragments of natural HIV co-receptors as described in Michael Farzan et al, (2000, J Biol Chem 27; 275(43):33516-21) and Lokesh A et al, (2004, Blood, 103(4):1211-7) and (Naiming Zhou and al, 2002, JBC) and Navid Madani et al, Small-Molecule CD4 Mimics Interact with a Highly Conserved Pocket on HIV-1 gp120, 2008 and Loïc Martin et al, Rational design of a CD4 mimic that inhibits HIV-1 entry and exposes cryptic neutralization epitopes, 2002).

The term “functional equivalent” of HIV co-receptors, as used herein, refers to a polypeptide that possesses a similar or identical function as a parent polypeptide but need not necessarily comprise an amino acid sequence that is similar or identical to that polypeptide. By a similar or identical function is intended the binding capacity to the viral particles, in particular to gp120-CD4 complex. Preferably, in the context of the present invention, a functional equivalent has an amino acid sequence that is at least about 30%, more preferably, at least about 40%, at least about 50%, at least about 60%, at least about 70%, at least about 80%, at least about 90%, at least about 95%, at least about 98%, at least about 99% identical to the amino acid sequence of the HIV co-receptors, and/or contains a characteristic sequence thereof. Moreover, those of ordinary skill in the art will understand that protein sequences generally tolerate some substitution without destroying activity. Thus any polypeptide that retains activity and shares at least about 30-40% overall sequence identity, often greater than about 50%, 60%, 70%, or 80%, and further usually including at least one region of much higher identity, often greater than about 90%, 96%, 97%, 98% or 99% in one or more highly conserved regions usually encompassing at least 3-4 and often up to 20 or more amino acids, with the parent polypeptide.

In one embodiment, the invention therefore provides conjugates comprising at least one CD36 targeting agent, preferably an antibody which recognizes, preferably specifically, and binds to CD36, associated to an anti-HIV effector moiety, wherein said anti-HIV effector moiety is selected from the group consisting of CCR5, CXCR4, CD4, T20, NBD-556, CD4M33, fragments thereof and functional equivalents thereof, preferably selected from the group consisting of CCR5, CXCR4, CD4, T20, NBD-556, and CD4M33, even more preferably selected from the group consisting of CCR5, CXCR4, CD4, and T20.

### Conjugation

The conjugate molecules of the invention may be formed using any techniques, in particular via chemical conjugation or genetic engineering.

In some conjugates of the present invention, the CD36 targeting agent is linked to the anti-HIV effector molecule via chemical conjugation, optionally via a linker. In the conjugates, the binding capacities of the CD36 targeting agent and the anti-HIV effector molecule are not substantially altered by the conjugation, meaning that they keep their binding ability.

The anti-CD36 binding molecule may be conjugated or linked to the molecule effector by a number of linkages including sulfide linkages, hydrazone linkages, hydrazine linkages, ester linkages, amido linkages, amino linkages, imino linkages, thiosemicarbazone linkages, semicarbazone linkages, oxime linkages, and carbon-carbon linkages. A sulfide linkage may be preferred, where the binding molecule may include disulfide linkages, which may be reduced to provide free thiol groups.

In particular, the anti-HIV effector may be linked to an antibody or other CD36 binding agent via an acid labile conjugation, or by a photolabile conjugation. The derivatives can be condensed with a peptide having a suitable sequence and subsequently linked to a cell binding agent to produce a peptidase labile conjugation. The conjugates can be prepared to contain a primary hydroxyl group, which can be succinylated and linked to a cell binding agent to produce a conjugate that can be cleaved by intracellular esterases to liberate free derivative. Preferably, the derivatives are synthesized to contain a free or protected thiol group, and then one or more disulfide or thiol-containing derivatives are each covalently linked to the cell binding agent via a disulfide bond or a thioether link.

Numerous methods of conjugation are taught in U.S. Pat. No. 5,416,064 and U.S. Pat. No. 5,475,092.

The molecules can be conjugated by a linker. Within a conjugate of the present invention, a linker spaces and covalently links together an anti-HIV effector and a CD36 targeting agent. A linker may be defined as a bifunctional chemical moiety into a normally stable tripartite molecule, releasing one of said chemical moieties from the tripartite molecule by means of cleavage; and following said cleavage, spontaneously cleaving the remainder of the molecule to release the other said spaced chemical moieties.

In certain embodiments of the present invention, said linker is chosen for its ability to be selectively cleaved at the target site or target cell, i.e., at or in the vicinity of the site of therapeutic action or activity of the chemotherapeutic agent. The cleavage may be enzymatic or chemical (e.g., reductive or pH conditions) in nature.

The spacer, preferable a cleavable spacer, is covalently linked at one of its ends to the CD36 targeting agent (or functional equivalent thereof) and covalently linked at its other end to the anti-HIV effector. Covalent binding between the spacer and CD36 targeting agent and between the spacer and effector molecule can be achieved by taking advantage of reactive functional groups already present on both molecules and/or spacer. Alternatively or additionally, reactive functional groups may be added to the anti-HIV effector to allow binding. Reactive functional groups may be selected from a wide variety of chemical groups including, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids, isonitriles; amidines, imides, imidates, nitrones, hydroxylamines, oximes, hydroxamic acids, thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Reactive functional groups also include those usually used to prepare bioconjugates, e.g., N-hydroxysuccinimide esters, maleimides and the like (see, for example, Hermanson, “Bioconjugate Techniques”, Academic Press: San Diego, 1996). Methods to introduce each of these functional groups are well known in the art and their application to or modification for a particular purpose is within the ability of one of skill in the art (see, for example, Sandler and Karo, Eds., “Organic Functional Group Preparations”, Academic Press: San Diego, 1989). Reactive functional groups may be protected or unprotected.

In some other embodiments, the conjugate is produced a fusion protein by recombinant engineering. Optionally, a spacer/linker can be introduced between the CD36 targeting agent and the anti-HIV effector. Such spacers are known to those skilled in the art. They usually comprise, or consist essentially of non polar amino acids such as Glycine, Serine or Alanine residues. Some of them derive from immunoglobulin amino acid sequences. In one specific embodiment, said spacers comprise or consists in a series of reiterated glycine and/or alanine residues, such as repetitions of Gly-Gly-Gly-Gly-Ser peptide or an analogous derived sequence. In particular, the spacer/linker can be cleavable.

This feature allows the anti-HIV agent that is relatively innocuous to cells while still in the conjugated form to be transported through the system without decomposition of the conjugate and to be delivered at the target site where, in the presence of the “cleaving” enzyme or conditions, it is selectively released to its pharmacologically active form. In this regard, a conjugate of the present invention acts as a prodrug. This aids in reducing systemic activation of the therapeutic agent, reducing toxicity, reducing side effects, and enhancing the efficacy of said agent by increasing its concentration at the target site.

Exemplary mechanisms by which cleavage of a spacer may release an anti-HIV effector from the CD36 targeting agent include hydrolysis in the acidic pH of lysosomes (hydrazones, acetals, and cis-aconitate-like amides), peptide cleavage by lysosomal enzymes (e.g., the capthepsins and other lysosomal enzymes), and reduction of disulfides (e.g., by glutathione). linkers whose cleavage is based on these different mechanisms have been designed and are known in the art. Examples of such linker spacers include, but are not limited to, those described in U.S. Pat. Nos. 5,773,001; 5,739,116; 5,877,296; 5,728,868; 5,770,731; 6,214,345; 6,218,519; 6,268,488; 7,091,186; 7,232,805; and 7,235,585; and PCT Publication No. WO 2005/0112919.

Thus, in certain embodiments, a conjugate of the present invention comprises at least one CD36 targeting agent, associated to an anti-HIV effector through a cleavable spacer, wherein the linker is hydrolysable in acidic pH. In other embodiments, the linker is enzymatically cleavable. In yet other embodiments, the spacer is cleavable by reductive conditions.

### Pharmaceutical Composition

The CD36 antagonist, the CD36 antibody or the conjugate described above, may be combined with pharmaceutically acceptable excipients.

The present invention relates to
a pharmaceutical composition comprising an antibody that recognizes and binds CD36, or an antigen-binding fragment or derivative thereof, or a CD36 antagonist, or a CD36 conjugate, in particular a conjugate comprising at least one CD36 targeting agent associated to at least one anti-HIV effector moiety, and optionally a pharmaceutically acceptable carrier, for use in the treatment of HIV infection, optionally in combination with another anti-viral treatment;
an antibody that recognizes and binds CD36, or an antigen-binding fragment or derivative thereof, or a CD36 antagonist, or a CD36 conjugate, in particular a conjugate comprising at least one CD36 targeting agent associated to at least one anti-HIV effector moiety, and optionally a pharmaceutically acceptable carrier, for use in the treatment of HIV infection.
a method for treating HIV infection in a subject in need thereof, comprising administering an effective amount of a pharmaceutical composition comprising an antibody that recognizes and binds CD36, or an antigen-binding fragment or derivative thereof, or a CD36 antagonist, or a CD36 conjugate, in particular a conjugate comprising at least one CD36 targeting agent associated to at least one anti-HIV effector moiety and optionally a pharmaceutically acceptable carrier;
a combined preparation, product or kit containing (a) an antibody that recognizes and binds CD36, or an antigen-binding fragment or derivative thereof, or a CD36 antagonist, or a CD36 conjugate, in particular a conjugate comprising at least one CD36 targeting agent associated to at least one anti-HIV effector moiety and (b) an anti-viral treatment as a combined preparation for simultaneous, separate or sequential use, in particular in the treatment of HIV infection; and
a method for treating HIV infection in a subject in need thereof, comprising administering an effective amount of a pharmaceutical composition comprising an antibody that recognizes and binds CD36, or an antigen-binding fragment or derivative thereof, or a CD36 antagonist, or a CD36 conjugate, in particular a conjugate comprising at least one CD36 targeting agent associated to at least one anti-HIV effector moiety, and an effective amount of a pharmaceutical composition comprising an anti-viral agent.

The form of the pharmaceutical compositions, the route of administration, the dosage and the regimen naturally depend upon the condition to be treated, the severity of the illness, the age, weight, and sex of the patient, etc.

The pharmaceutical or therapeutic compositions of the invention can be formulated for a topical, oral, parenteral, intranasal, intravenous, intramuscular, subcutaneous or intraocular administration and the like.

The antibody that recognizes and binds CD36, or the antigen-binding fragment or derivative thereof, or the CD36 antagonist, or the CD36 conjugate, used in the pharmaceutical composition of the invention is present in a therapeutically effective amount. The term “therapeutically effective amount” as used in the present application is intended an amount of therapeutic agent, an antibody that recognizes and binds CD36, or an antigen-binding fragment or derivative thereof, or a CD36 antagonist, or a CD36 conjugate, administered to a patient that is sufficient to constitute a treatment of HIV infection as defined above.

The pharmaceutical composition of the invention is formulated in accordance with standard pharmaceutical practice (Lippincott Williams & Wilkins, 2000 and Encyclopedia of Pharmaceutical Technology, eds. J. Swarbrick and J. C. Boylan, 1988-1999, Marcel Dekker, New York) known by a person skilled in the art.

For oral administration, the composition can be formulated into conventional oral dosage forms such as tablets, capsules, powders, granules and liquid preparations such as syrups, elixirs, and concentrated drops. Non toxic solid carriers or diluents may be used which include, for example, pharmaceutical grades of mannitol, lactose, starch, magnesium stearate, sodium saccharine, talcum, cellulose, glucose, sucrose, magnesium, carbonate, and the like. For compressed tablets, binders, which are agents which impart cohesive qualities to powdered materials, are also necessary. For example, starch, gelatine, sugars such as lactose or dextrose, and natural or synthetic gums can be used as binders. Disintegrants are also necessary in the tablets to facilitate break-up of the tablet. Disintegrants include starches, clays, celluloses, algins, gums and crosslinked polymers. Moreover, lubricants and glidants are also included in the tablets to prevent adhesion to the tablet material to surfaces in the manufacturing process and to improve the flow characteristics of the powder material during manufacture. Colloidal silicon dioxide is most commonly used as a glidant and compounds such as talc or stearic acids are most commonly used as lubricants.

For transdermal administration, the composition can be formulated into ointment, cream or gel form and appropriate penetrants or detergents could be used to facilitate permeation, such as dimethyl sulfoxide, dimethyl acetamide and dimethylformamide.

For transmucosal administration, nasal sprays, rectal or vaginal suppositories can be used. The active compound can be incorporated into any of the known suppository bases by methods known in the art. Examples of such bases include cocoa butter, polyethylene glycols (carbowaxes), polyethylene sorbitan monostearate, and mixtures of these with other compatible materials to modify the melting point or dissolution rate.

Pharmaceutical compositions according to the invention may be formulated to release the active drug substantially immediately upon administration or at any predetermined time or time period after administration.

Pharmaceutical compositions according to the invention can comprise one or more antibodies that recognizes and binds CD36, or antigen-binding fragments or derivatives thereof, or one or more CD36 antagonists, or one or more CD36 conjugates associated with pharmaceutically acceptable excipients and/or carriers. These excipients and/or carriers are chosen according to the form of administration as described above. Other active compounds can also be associated with CD36 inhibitors, CD36 antibodies or CD36 conjugates, in particular antiviral drugs such as the ones used in anti-HIV therapy, preferably used in HAART therapy including the ones disclosed above. The initiation of the HAART may occur before, after or concurrently with administering a therapeutically effective amount of a composition according to the invention.

Such combination with other antiviral treatments is aimed at reinforcing the desired effects. Indeed, adding a CD36 antagonist, antibody or conjugate to such an anti-HIV therapy allowing a significant decrease of the viral load should allow to clear viral reservoirs in macrophages, with the aim of eradicating the virus.

The amount of CD36 inhibitor, antibody or conjugate to be administered has to be determined by standard procedure well known by those of ordinary skill in the art. Physiological data of the patient (e.g. age, size, and weight), the routes of administration and the disease to be treated have to be taken into account to determine the appropriate dosage. The CD36 antagonist, antibody or conjugate may be administered as a single dose or in multiple doses. If the CD36 antagonist is a small molecule inhibiting the CD36 activity, each unit dosage may contain, for example, from 200 to 1000 mg/kg of body weight, particularly from 500 to 800 mg/kg of body weight. If the CD36 antagonist is an anti-CD36 antibody, each unit dosage may contain, for example, from 0.1 to 20 mg/kg of body weight, particularly from 4 to 10 mg/kg of body weight. If the CD36 antagonist is a CD36 RNAi molecule, each unit dosage may contain, for example, from 2 to 50 mg/kg of body weight, particularly from 5 to 20 mg/kg of body weight. If the composition comprises an anti-CD36 antibody, antagonist or not, or a CD36 conjugate, each unit dosage may contain, for example, from 0.1 to 20 mg/kg of body weight, particularly from 4 to 10 mg/kg of body weight.

### Use of the Composition of the Invention.

The inventors showed that inhibition of CD36 led to a strong reduction of HIV virions production by macrophages, and thus to a diminution of HIV viral load in the supernatant. They further showed that the anti-CD36 exposure induces virions tethering at the site of viral assembly and thus their retention within the VCCs of macrophages.

The invention thus relates to
a method for treating HIV infection by administering a therapeutically effective amount of an antibody that recognizes and binds CD36, or an antigen-binding fragment or derivative thereof, or a CD36 antagonist, or a CD36 conjugate, in particular a conjugate comprising at least one CD36 targeting agent associated to at least one anti-HIV effector moiety,
the use of an antibody that recognizes and binds CD36, or an antigen-binding fragment or derivative thereof, or a CD36 antagonist, or a CD36 conjugate, in particular a conjugate comprising at least one CD36 targeting agent associated to at least one anti-HIV effector moiety for treating HIV infection in patients in need thereof, and
the use of an antibody that recognizes and binds CD36, or an antigen-binding fragment or derivative thereof, or a CD36 antagonist, or a CD36 conjugate, in particular a conjugate comprising at least one CD36 targeting agent associated to at least one anti-HIV effector moiety, for preparing a medicament for treating HIV infection in patients in need thereof.

In particular, the treatment allows the improvement of the clinical outcome of a HIV-infected patient, through the diminution of HIV viral load. The composition according to the invention may be administered prior to the onset of the disease, for a prophylactic or preventive action. Alternatively or additionally, the composition according to the invention may be administered after initiation of the disease or condition, for a therapeutic action. In particular, the composition of the invention may be used in combination with another anti-viral treatment or anti-HIV therapy, in particular in combination with highly active retroviral therapy (HAART). The composition of the invention may be administered before, during or after said other anti-HIV therapy, preferably after said anti-HIV therapy to clean up HIV reservoirs.

The term “anti-HIV therapy” as used herein means any anti-HIV drug found useful for treating HIV infection in human alone, or as part of multidrug combination therapies, especially the triple and quadruple combination therapies called HAART. Typical suitable anti-HIV therapies include, but are not limited to multidrug combination therapies such as at least those described in EP 1034790 A2.

The term “highly active antiretroviral therapy” or HAART as used herein means the multi-drug therapies used in current clinical treatment of HIV infections, including but not limited to the multi-drug therapies, e.g., the triple and quadruple drug therapies such as disclosed by A-M. Vandamme et al., in Antiviral Chemistry & Chemotherapy, 9: 187-203 (1998) which describes the current clinical treatments of HIV infections, including when to start multi-drug therapy and which drugs to combine. The triple drug therapy may include two nucleoside and nucleotide reverse transcriptase inhibitors (“NRTIs”) and one protease inhibitor (“PI”), but there are many issues to be considered in the choice of the precise HAART for any patient. The highly active antiviral therapy (HAART) usually includes a combination of reverse transcriptase (RT) and protease inhibitors that induce undetectable levels of viral RNA in the peripheral blood plasma. In a particular embodiment, the composition of the invention is used in combination with HAART comprising a combination of one or several reverse transcriptase inhibitors, one or several protease inhibitors, and/or one or several integrase inhibitors. The reverse transcriptase inhibitor(s) may be selected from the group consisting of deoxythymidine, zidovudine, stavudine, didanosine, zalcitabine, abacavir, lamivudine, emtricitabine, entecavir, apricitabine, adefovir, efavirenz, nevirapine, delavirdine, etravirine, rilpivirine and tenofovir. The integrase inhibitor(s) may be selected from the group consisting of raltegravir, elvitegravir and MK-2048. The protease inhibitor(s) may be selected from the group consisting of lopinavir, indinavir, nelfinavir, amprenavir, ritonavir, saquinavir, atazanavir, fosamprenavir, tipranavir and darunavir. Preferably, HAART comprises a combination of three or four of these inhibitors. In particular HAART may comprise two reverse transcriptase inhibitors, one protease inhibitor, and optionally one integrase inhibitor.

The term “HIV infection” defines a condition caused by the human immunodeficiency virus (HIV). The subject may be infected by HIV-1 or HIV-2 virus, or both. Preferably, the subject is infected with HIV-1. Infection with HIV-1 is associated with a progressive decrease of the CD4⁺ T cell count and an increase in viral load. The stage of infection can be determined by measuring the patient's CD4⁺ T cell count, and the level of viral load in the blood. HIV infection has four basic stages: incubation period, acute infection, latency stage and AIDS. The initial incubation period upon infection is asymptomatic and usually lasts between two and four weeks. The second stage, acute infection, lasts an average of 28 days and can include symptoms such as fever, lymphadenopathy, pharyngitis, rash, myalgia, malaise, and mouth and esophageal sores. The latency stage, which occurs third, shows few or no symptoms and can last anywhere from two weeks to twenty years and beyond. AIDS, the fourth and final stage of HIV infection, is characterized by a very low level of CD4+ T cells and a severe immunodeficiency, and is accompanied by various opportunistic infections. “HIV viral load” tests are reported as the number of HIV copies in a milliliter (copies/mL) of blood. If the viral load measurement is high, it indicates that HIV is reproducing and that the disease will likely progress faster than if the viral load is low. During treatment and monitoring, a high viral load can be anywhere from 5,000 to 10,000 copies/mL. Initial, untreated, and uncontrolled HIV viral loads can range as high as one million or more copies/mL. A low viral load is usually between 40 to 500 copies/mL, depending on the type of test used. This result indicates that HIV is not actively reproducing and that the risk of disease progression is low. A viral load result that reads “undetectable” does not mean that you are cured. It may mean that either the HIV RNA is not present in your blood at the time of testing or that the level of HIV RNA is below the threshold needed for detection. Even though HIV may be undetectable in the blood, it persists in cells and tissues throughout the body as “HIV provirus”. HIV provirus refers to virus that has moved into cells and into the nucleus, where it has become integrated with the DNA of the host cell. This is also call “HIV proviral DNA”. Change in viral load is also a very important measurement. A rising count indicates either that the infection is getting worse or that you have developed resistance to the drugs that are being used for therapy, while a falling count indicates improvement and suppression of the HIV infection. Good clinical practice to minimize detectable HIV RNA plasma levels are known to those skilled in the art. See for example A-M. Vandamme et al., in Antiviral Chemistry & Chemotherapy, 9: 187-203 (1998) and “Drugs for HIV Infection” in The Medical Letter Vol. 39 (Issue 1015) Dec. 5, 1997, pages 111-116.

Methods of Identifying Compounds Useful for the Treatment and/or Prevention of HIV Infection.

Of particular interest, the inhibition ability of a CD36 inhibitor according to the invention may be further assessed for its ability to prevent HIV virions release, preferably in macrophages, by any technique known to those skilled in the art including those described in the Example section of the present specification.

In addition, the present invention relates to the use, preferably in vitro or ex vivo, of the CD36 receptor as a screening tool for compounds useful for the treatment and/or prevention of HIV infection.

The invention thus also relates to a method for screening for compounds useful for the treatment of HIV infection, said method comprising determining whether a candidate compound recognizes and binds to CD36 or reduces or inhibits expression of CD36, wherein the ability of said candidate compound to recognize and bind to CD36 or to reduce or inhibit expression of CD36 is indicative of the usefulness of said candidate for the treatment of HIV infection.

The invention further relates to a method for screening for compounds useful for the treatment of HIV infection, said method comprising determining whether a candidate compound inhibits the activity or expression of CD36, wherein the ability of said candidate compound to inhibits the expression or activity of said CD36 is indicative of the usefulness of said candidate compound for the treatment of HIV infection.

In particular, the invention is drawn to a method for screening for a compound for the treatment of HIV infection comprising:
a) providing or obtaining a candidate compound; and
b) determining whether said candidate compound is a CD36 antagonist;
   wherein said determination that said candidate compound is a CD36 antagonist indicates that said candidate compound is a compound suitable for the treatment of HIV infection.

The candidate compound to be tested in the frame of this method may for example be to a chemical molecule (preferably a small molecule), an antibody, a peptide, a polypeptide, an aptamer, an iRNA, a sense or antisense oligonucleotide, or a ribozyme.

The screening method according to the invention may further comprise testing the candidate compound to a cellular model or administering to a non-human animal model of HIV infection in order to confirm that the candidate compound is suitable for the treatment of HIV infection. Such cellular models and non-human animal model of HIV infection are known to the skilled in the art and include cellular models such as DC/macrophages/monocytes or DC/macrophage/monocyte cell lines infected with HIV, and primate models such as Rhesus Monkeys or SIVmac239.

The invention also pertains to a method for identifying a compound for the treatment of an HIV infection comprising:
a) providing or obtaining an antagonist of the CD36 receptor;
b) administering said CD36 antagonist to a non-human animal model of HIV infection, and
c) determining whether said CD36 antagonist decreases CD36 surface expression into said model,
   wherein the determination that said antagonist decrease CD36 surface expression of said animal model, compared to a control animal, indicates that said antagonist is a compound for the treatment of HIV infection.

Once a compound for the treatment of HIV infection has been identified, a pharmaceutical composition comprising the compound and a pharmaceutically acceptable carrier may be manufactured. The pharmaceutical composition may then be tested in order to confirm its efficacy, e.g. during a quality control process and/or during optimization of the formulation of the pharmaceutical composition.

Therefore, the invention also pertains to a method for manufacturing a pharmaceutical composition comprising an antagonist of the CD36 receptor, wherein said method comprises the steps of:
a) producing said pharmaceutical composition;
b) administering said pharmaceutical composition to a non-human animal model infected with HIV.
c) Optionally, determining whether the administration of said pharmaceutical composition decreases CD36 surface expression of said animal model, compared to a control animal.

Where the term “comprising” is used in the present specification, this can preferably be replaced by “consisting essentially of”, or by “consisting of”.

The present invention will be further illustrated by the additional description which follows, which refer to examples illustrating that CD36 expression is critical for efficient HIV-1 release and that CD36 specific antibodies are specifically internalized into VCC and modulate HIV-1 release from macrophages. It should be understood however that the invention is defined by the claims, and that these examples are given only by way of illustration of the invention and do not constitute in anyway a limitation thereof

### Examples

### Materials and Methods

### Plasmids, Antibodies and Reagents

pVSV-G (Becton Dickinson) pEGFP N1 (Clontech), psPax2 (addgene number 12260) plasmids were used. pUC NL4-3 Gag-iGFP T-tropic envelope was a gift from B. Chen (Hubner et al., 2007) and was used to derive the HIV-1 Gag-iGFP which carries a R5-tropic envelope with the V3-loop V92th014.12 (Koppensteiner et al., 2011) and pHIV-1 Gag-iGFPΔEnv were provided by Michael Schindler (Munich, Germany). The HIV-1 Gag-imCherry was obtained by sub-cloning the region from NarI to BamHI from the Gag-iGFP into pUNO-mcs vector (InvivoGen). Then, the GFP was replaced with the mCherry sequence amplified with the primers mCherry_F_MluI (5′ tgtacaaacgcgtGTGAGCAAGGGCGAGGAG 3′ SEQ ID No 4) and mCherry_R_XbaI (5′ ttggcttctagaCTTGTACAGCTCGTCCATGC 3′ SEQ ID No 5) using the restriction sites XbaI and MluI. The fragment obtained, containing the mCherry in place of the GFP was then re-cloned in the HIV-1 Gag-iGFPΔEnv backbone using BssHII and SpeI. To build the pDEST DH1 CD36, the CD36 cDNA from the pDONR221 CD36 (DNASU reference HsCD00045106) was cloned with Gateway technology (Invitrogen) into a pDEST DH1, which is a modified version of pCDH1 (System Bioscience) in which the MCS has been replaced by a gateway cassette. To construct CD36-GFP, pDONR221 CD36 was used as a template to amplify the CD36 cDNA with the primers:

### CD36 N1 F (5′ ACCGCGGGCCCGGGATATGGGCTGTGACCGGAACTG 3′ SEQ ID No 6) and

CD36 N1 R (5′ CATGGTGGCGACCGGTGGTTTTATTGTTTTCGATCTGCATGCAC 3′ SEQ ID No 7). The PCR product was then cloned into the pEGFP N1 using the restriction sites XmaI and AgeI. The resulting cDNAs CD36-GFP were cloned with Gateway technology (Invitrogen) into a pDEST DH1 to obtain the pDEST DH1 CD36-GFP. CD36 mutants were produced with the QuikChange II Site-Directed Mutagenesis Kit (Agilent Technologies) using the pEXP DH1 CD36 as template. Primers were designed with the QuikChange® Primer Design Program (https://www.genomics.agilent.com/CollectionSubpage.aspx?PageType=Tool& SubPageType=ToolQCPD&PageID=2296).

Goat polyclonal antibodies anti-p24, mouse monoclonal antibodies specific for human CD81 (clone TS81), CD68 (clone KP1), SRA1 (clone 7H1G1), rabbit antibodies specific for human LDLR (clone EP1553Y) and for Apolipoprotein B (ab20737) were purchased from Abcam. Rabbit polyclonal antibodies anti-SR-B1 (NB400-104, Novus Biologicals) and anti-CD9 (clone H-110, Santa-Cruz), mAbs specific for CD9 (MEM-61, Immunotools), ICAM-1 (clone HA58, BD Biosciences), CD36 (clones CLB-IVC7 and TR9 from Immunotools and clone FA6-152 from StemCell Technologies), FITC or PE-labeled (ImmunoTools), unlabeled isotype controls (BD bioscience), secondary antibodies conjugated with Alexa Fluor 488 or 647 (Invitrogen), Cy3 or Cy5 (Jackson) were used. Isotype controls FITC- and PE-labeled as well as the anti-p24 (KC57-FITC or KC-57 RD1) were purchased from Beckman Coulter whereas FITC- and PE-labeled anti-CD36 (CLB-IVC7 and TR9) were purchased from Immunotools and Acris Antibodies. Human antibodies specific for EGFP and rabbit antibodies anti-α-Tubulin were kindly given by A. Marjou (Institut Curie). The Phospho-SAPK/JNK (Thr183/Tyr185) (81E11) Rabbit mAb from Cell Signaling was used. Rabbit polyclonal antibodies specific for p17 as well as p55 (NIH AIDS reagents 4811 from P. Spearman) and HIV Immunoglobulins (used at 0.37 mg/ml in the infectivity assay on TZM-bl cells) were obtained through the NIH AIDS Research and Reference Reagent Program, Division of AIDS, NIAID, NIH. Fab fragments were produced using the Mouse IgG1 Fab and F(ab′)2 Preparation Kit from Thermo Scientific. The human FcR Blocking Reagent (Miltenyi Biotec) was tested by FACS for its capacity to stain the plasma membrane of primary macrophages and was used according to the manufacturer's guidelines. TSP-1 was purchased from (Bruna) whereas Collagen type I was purchased from Sigma. The inventors also used DiI-MOx-LDL labeled (Kalen Biomedical), dextran coupled to Alexa Fluor® 647 10,000 MW (Molecular Probes), IL2 (eBioscience), PHA-L (Sigma), AZT (Sigma), and M-CSF (Calbiochem) dissolved in ultrapure water (25 μM stock).

### Primary Cells and Cell Lines

PBMC purified from blood from healthy donors were used to isolate monocytes by positive selection using CD14+ microbeads (Miltenyi). Monocytes were differentiated for 7 days into macrophages by culture on non-treated culture plastic (Nunc) in RPMI 1640 supplemented with 10% FCS (Gibco) and M-CSF (25 μg/ml). Ghost, HeLa, HEK293T (ATCC CRL-11268) and TZM-Bl cell lines were cultured in complete DMEM Glutamax. HeLa-CD36 cells were produced by lentiviral transdunction of the CD36 gene. Puromycin selection (Invivogen at 4 μg/ml) was performed to obtain a pure population of transduced cells. HeLa as well as HeLa cells expressing the different CD36 mutants were cultured in DMEM with Glutamax completed with 10% FCS (Gibco) and 1% Penicillin-Streptomycin (Gibco).

### Virus Preparations and Infections

The following virus strains have been produced and used in the present study: NL4-3, NL(AD8) (NLAD8), HIV Gag-iGFP, HIV Gag-iGFP ΔEnv and HIV Gag-imCherry ΔEnv. Two lentiviral vectors have been produced; pDEST DH1 CD36 and pDEST DH1 CD36-GFP. Viruses were produced by co-transfection of the appropriate proviral cDNA, the packaging psPax2 plasmid and eventually the pVSV-G plasmid (when pseudotyping was required) into 293T cells using GeneJuice (Novagen). Supernatants were harvested 72 hr after transfection, filtered and stored at −80. Virus preparations were titrated by infecting the Ghost reporter cell line and infectivity was measured 24 hr post-infection by flow cytometry as described (Morner et al, 1999) SIV(VLP) were produced and used as described (Manel et al, 2010). NLAD8-infected macrophage samples were prepared for immuno-electron microscopy as previously described (Jouve et al, 2007). For infection experiments, macrophages were infected at a Multiplicity of Infection (MOI) of 0.2 and kept in culture for indicated times. HeLa cells were infected at MOI 1 or 0.2 depending on the experiment. The 132W primary isolate (used at 10 ng of p24 for 1×10⁵ macrophages) was a gift of Olivier Schwartz, (Pasteur Institute, France).

### Viral Production and Infectivity Assays

Measure of HIV p24 was performed using the Innotest HIV Ag mAb Screening kit (Ingen).

Cell viability was evaluated using the CellTiter Glo (Promega).

For infectivity assays, virion-containing supernatants were normalized for equal amounts of p24 (final concentration of 10 ng/ml) and infectious titers were determined using TZM-bl indicator cells as described (Martin-Serrano et al., 2001). β-Galactosidase activity in the cell lysates was measured using the Gal-Screen® System for Mammalian Cells (Applied Biosystems).

### RNA Interference

The sequences of the various siRNA used in the present study are given in Table 1 (SEQ ID Nos 1-3). siRNA (100 nM final) were diluted in 250 μl of OptiMEM (Gibco) mixed with Sul of Interferin (Polyplus) and left at room temperature for 15 min. Complexes were added drop-wise onto the macrophage cultures. Cells were assayed 3 days later. Knock down efficiency was checked by flow cytometry.

TABLE 1

| | siRNA sequence | SEQ ID No |
|---|---|---|
| Luc | CGUACGCGGAAUACUUCGATT | 8 |
| Tsg101 | CAGUUUAUCAUUCAAGUGUAA | 9 |
| CD36_1 | GAACCUAUUGAUGGAUUAATT | 1 |
| CD36_2 | CCUUCACUAUCAGUUGGAATT | 2 |
| CD36_3 | GCAACAUUCAAGUUAAGCATT | 3 |

### Immuno Fluorescence Electron Microscopy and Live Imaging

Cells were fixed in 4% PFA for 15 min and permeablized in 0.05% saponin, 0.2% BSA in PBS. Antibody staining and washes were performed in the same buffer, before mounting coverslips in DAPI Fluoromount G (SouthernBiotech). Samples were then imaged on a Nikon Ti Inverted Microscope fitted with a confocal AIR system, using a 60× oil immersion objective with a numerical aperture of 1.4. Confocal images were collected as snapshot or 3D stacks with a focal step size of 0.5 μm. NIS Element, Methamorph and ImageJ software were used for image processing. No filter or background subtraction was used. Live imaging was performed using a Nikon Biostation IM-Q using a 20× objective or a Nikon Ti Inverted

Microscope fitted with a video-rate confocal system consisting of a spinning disk confocal head (Yokogawa).

For antibody feeding assay, cells were pre-cooled at 4° C. for 20 min before replacing the medium with ice-cold medium containing anti-CD36 (IVC-7) or the IgG1 isotype control.

Cells were kept at 4° C. or moved to 37° C. for 2 hours depending on the condition before fixation and staining

### Image Quantification

For quantification of the Pearson's coefficient, 3D stacks (z=0.5 μm) of infected macrophages were acquired with a Nikon AIR confocal system and analyzed with the NIS Element Ar software. For each cell a background of 100 for each channel was subtracted and plans containing no signal were discarded. Then the Pearson's coefficient of each plan of the stack was calculated, generating mean Pearson's coefficient per cell.

To quantify the total volume of VCCs per cell, the total intensity of Gag into VCCs per cell and the number of VCCs per cell, confocal plans with a z-step of 0.25 μm were acquired for more than 134 cells per condition. VCCs were 3D-reconstructed and analyzed in a blind manner using IMARIS 7.2.3 software. Gag fluorescence intensity corresponding to the VCCs was detected, smoothed at 0.15 μm and a background subtraction was applied based on largest sphere diameter (0.8 μm) criteria. Automatic segmentation was performed based on a region growing estimated diameter of 0.8 μm. Then, objects were filtered to take into account only VCCs above 0.050 μm3. Automated statistical analysis was run to extract the different parameters for each condition.

### Immunoblotting

Analyses were carried out as described (Gaudin et al., 2012) excepted for pJNK assay where lysis buffer was supplemented with NaF 5 mM and b-glycerophosphate 1 mM.

### CD36 mAb Blocking Assay

Infected macrophages were washed extensively and exposed to anti-CD36 mAb (FA6-152 or IVC-7) or the IgG1 isotype control at 37° C. for various periods of time. Supernatant was collected and analyzed for p24 content by ELISA. Intracellular quantification of p24 was performed by ELISA on cells lysed in the cell titer Glo buffer for 2 hours at 4° C.

### Macrophage-to-T-Cell HIV Transmission Assay

Macrophages (10⁵ cells/well) were infected with VSV-G-pseudotyped NL4-3 at MOI 1.5, washed extensively 7 days later and received 1.5 10⁵ CD4+ T lymphocytes/well. These CD4+ T lymphocytes were purified by negative selection (Miltenyi) from heterologous PBMCs that had been activated 48 hr before in RPMI, 10% FCS, 2.5 μg/ml PHA-L, 30 U/ml IL2. AZT (25 μM) was added at 0 hr (as a negative control) or 24 hr after the beginning of the co-cultures. At 48 hr, CD4+ T cells were collected, washed, fixed with PFA 1%, permeabilized, stained for Gag with KC57-FITC mAb, and analyzed by flow-cytometry in a BD Accuri C6 flow cytometer.

### Correlative Light-Electron Microscopy

Purified monocytes were plated with M-CSF on CELLocate coverslips (Eppendorf) that etched grids with coordinates, allowing the cell of interest to be found through all of the steps of the procedure. After 7 days, macrophages were infected with the CD36-GFP lentiviral vector and SIV(VLP). 7 days later, cells were infected with HIV Gag-imCherry ΔEnv. At 7 days post HIV infection, cells were imaged by spinning disk confocal microscopy, before fixation in 2.5% glutaraldehyde in 0.1M Cacodylate buffer. Then, coverslips were embedded in epon and processed for electron microscopy {Jouve, 2007 cell Host Microbe 2(2):85-95}.

### Results

### The Virus-Containing Compartment of HIV-1-Infected Macrophages Contains CD36 but not Other LDL Receptors

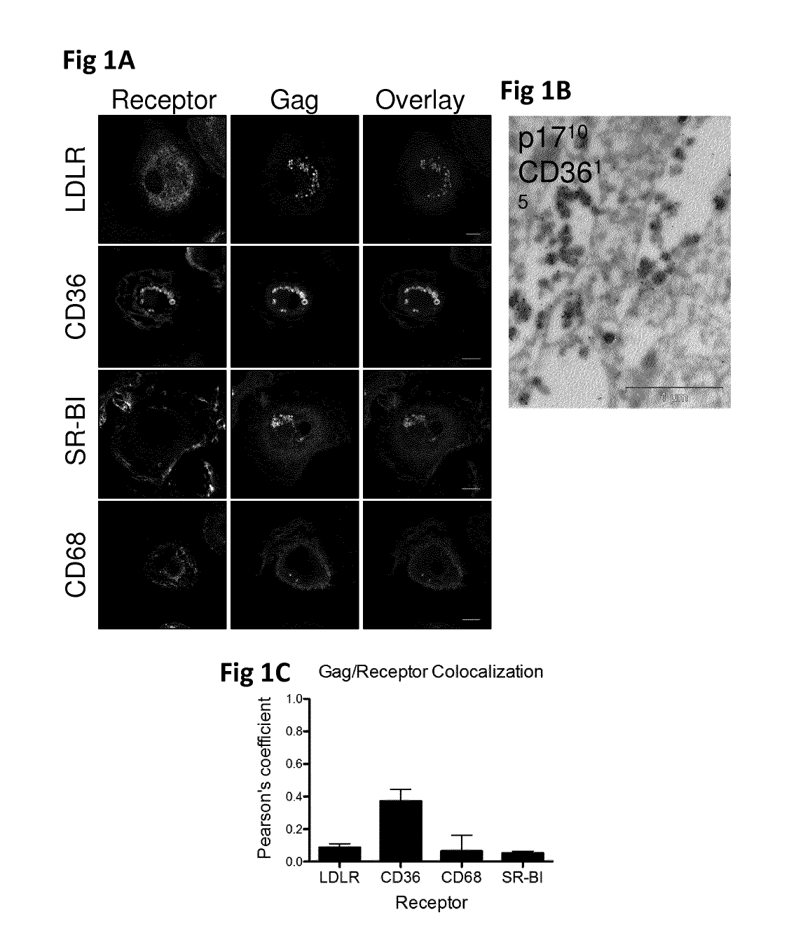

The inventors tested in HIV-1-infected macrophages the intracellular localization of receptors that are known to possess affinity for various LDL (May Kathryn et al, 2006, Uptake Arterioscler Thromb Vasc Biol, 26:1702-1711 and David R Greaves et al, Recent insights into the biology of macrophage scavenger receptors, 2004, JLR). The inventors analysed the intracellular distribution of 4 lipid receptors, for which specific antibodies were available, by immunofluorescence with respect to Gag localization in HIV-1-infected macrophages at 7 days post-infection (dpi) (FIG. 1A). Similar results were obtained when monitoring Env instead of Gag localization (not shown). The inventors observed that staining specific for SR-B1, CD68 and LDLR did not overlap with the one of Gag. The same held true for MSRA1 receptor (not shown). In contrast, CD36 staining was present at the plasma membrane level in membrane ruffles, but also in internal compartments that stained positive for Gag (FIG. 1A). Analysis at the ultra-structural level by immuno-EM of infected macrophages revealed the presence of CD36 in internal viral Gag+ viral particles present in the lumen of VCCs (FIG. 1B).

The inventors conclude that CD36 is associated with the VCCs in HIV-1-infected macrophages.

### HIV-1 CD36+ Compartments are Connected to the Plasma Membrane

Next, the inventors sought to better characterize the CD36+ compartments in infected macrophages. To be able to perform live cell imaging, the inventors built a lentivector encoding CD36-GFP (this fusion protein is known to be functional (Jianshe et al, 2007, Lipids in Health and desease, 6:24). In addition, the inventors checked that the distribution of CD36-GFP totally overlaps with the one of endogenous CD36 in macrophages (FIG. 12). As expected, confocal analysis of primary macrophages co-infected with this vector and HIV-1 Gag-imCherry revealed overlapping distribution of both fusion proteins in intracellular compartments (FIG. 2A).

One of the shared features between VCCs of infected macrophages and the lipid storage compartment in non-infected cells is their connection to the plasma membrane via microchanels (ref macrophages Maxfield) Welsch S, et al. (2007) PLoS Pathog 3, e36. Deneka M, et al. (2007) J Cell Biol 177, 329-341.

The inventors therefore tested the presence of such connections HIV-1-infected macrophages using a fixation-free approach combining correlative light and electron microscopy. Macrophages were infected with the lentivector encoding CD36-GFP and 5 days later with HIV-1 Gag-imCherry. Seven days later macrophages were imaged live before and immediately after adding a 10 kD Dextran-Alexa 647 (Dex-647) into the culture medium. Analysis of the images acquired by spinning disk microscopy revealed that Dex-647 was present in certain CD36-GFP+ Gag-imCherry+ compartments (FIG. 2A). Moreover, correlative EM on the very same cell established that such compartments were true VCCs as they contained both mature and immature viral particles as well as budding profiles at the limiting membrane of the compartment (FIG. 2B). Note that these results also show that CD36-GFP does not form aggregates, behaves like endogenous CD36 and confirm that HIV Gag-iCherry is suitable for time-lapse studies in macrophages.

To evaluate whether related compartments exist in normal macrophages, the inventors analyzed uninfected macrophages expressing CD36-GFP by confocal microscopy. The fusion protein was also found at the plasma membrane and in internal compartments that stained positive for CD81 (FIG. 2C). Finally, such CD36 compartments were also immediately accessible to Dextran indicating that they can be connected to the plasma membrane (FIG. 2D).

The inventors conclude that, in HIV-1-infected as well as in non-infected macrophages, CD36+CD81+ compartments are accessible to the extracellular medium. Moreover, in the case of HIV-1-infected macrophages, these compartments do contain viral particles and are thus genuine VCCs.

### Gag+ Compartments are Formed on Preexisting CD36+ Compartments

The fact that CD36+ compartments in normal macrophages and VCCs in infected ones share several important features (presence of specific markers; CD36, CD9, CD81, and connection with the plasma membrane) suggested that HIV-1 makes usage of preexisting CD36+ compartments to convert them into VCCs. To test this hypothesis, primary macrophages, infected with the CD36-GFP lentivector and 5 days later with HIV Gag-iCherry, were analyzed 0.5 day later by time-lapse epifluorescent microscopy during 7 days (one image every 15 min), (FIG. 3). CD36-GFP expression was observed in many cells that did not express detectable levels of Gag. CD36-GFP was present at the plasma membrane but also into intracellular compartments. With time, a diffuse Gag-iCherry signal in the cytosol started to appear. It then became concentrated into internal compartments, most of them being CD36-GFP+(FIG. 3).

Taken together, these data indicate that Gag accumulates in preexisting CD36+ compartments of infected macrophages where new viral particles are formed.

### HIV-1 Release by Macrophages is Dependent on CD36 Expression

To further establish the role of CD36 during the HIV-1 cycle in macrophages, the inventors sought to determine whether CD36 silencing affects viral production in HIV-1-infected macrophages. The inventors thus performed HIV-1 infection of primary macrophages followed by transfection of siRNAs specific for CD36, luciferase as a negative control, or TSG101 as a positive one, following the experimental design schematized in FIG. 4A. Efficiency of CD36 silencing ranged from 50 to 70% as determined by intracellular FACS analysis (FIG. 4D). Cell viability (FIG. 4C) was similar in the different cell populations upon viral infection and siRNA transfection. Interestingly, CD36 silencing using 3 different siRNAs specific for CD36 led to a strong reduction (up to 65%) of the amount of p24 secreted in the supernatant, which reflects the secretion of viral particles (FIG. 4B). The extent of the inhibition was similar to that obtained when the ESCRT (Endosomal Sorting complexes Required for Transport) protein Tsg101, which is crucial for viral budding (Garrus et al., 2001, Cell 107(1):55-65), was knocked down. The kinetics of the inhibitory effect of CD36 depletion was analysed. The amount of p24 released during 24 hours by the different cell populations was determined at 3 different times post-infection and compared to the amount found in the corresponding cell lysates collected at day 4 (FIG. 4E). The supernatant p24 levels increased with time post infection as expected while the inhibitory effect of CD36 silencing was visible at day 2-3 and 3-4.

The inventors conclude that silencing of CD36 inhibits the release of HIV-1 particles by infected macrophages.

Targeting VCCs in Macrophages with a CD36-Specific mAb

The present results showing that CD36 is associated with viral particles (FIG. 1B), suggested that CD36, like its ligand, can be internalized and reach VCCs. To test this hypothesis, HIV-1-infected macrophages were exposed to a mAb specific for CD36 at 37° C. for 2 hours. After fixation and appropriate staining, cells were analyzed by confocal microscopy (FIG. 5A). The CD36-specific mAb did access Gag+CD9+ compartments, whereas an isotype control used in the same conditions did not. Exposure at 4° C. instead of 37° C. prevented the CD36 mAb to access the VCCs (FIG. 5A). Moreover, simultaneous exposure of HIV-1-infected macrophages to antibodies specific for SR-B1 and CD36 at 37° C. for 2 hours led to the specific accumulation of the CD36 mAb into Gag+ compartments (FIG. 5B). In contrast, the SR-B1 antibodies were also internalized but in intracellular compartments clearly distinct from the Gag+ ones. Such transport did not occur at 4° C. for both types of antibodies.

These data suggested that anti-CD36 mAb bound to surface displayed CD36 can be actively and specifically transported into internal VCCs in HIV-1-infected macrophages.

### Exposure of Infected Macrophages to a CD36-Specific mAb Inhibits HIV-1 Release

Infected macrophages are thought to represent a permanent threat able to release their internal infectious viral stock at any time. The fact that CD36 expression is highly restricted to macrophages and to a few other cell types suggests that targeting this molecule can be of interest for therapeutic purposes (FIG. 11). Moreover, the fact that exogenously added CD36-specific mAb can access VCCs prompted us to test the effect of such mAbs on HIV-1 release by infected macrophages (see experimental design FIG. 6A).

Exposure of HIV-1-infected macrophages from two different donors to CD36-specific mAb for 24 hours led to a strong reduction of the amounts of p24 released as compared to cultures exposed to an isotype control (FIG. 6B). Conversely, the amounts of p24 detected in the cell lysates were increased in anti-CD36 treated cultures as compared to isotype treated culture (FIG. 6C). As a result the sums of p24 amounts found in the supernatant and cell lysate in each of the condition remained roughly constant (FIG. 6D). The infectivity of the virus released in both conditions remained similar (FIG. 6E). This result was confirmed using viral particles produced by macrophages and exposed to anti-CD36 mAb or its isotype control just before being added onto TZM-bl reporter cells (not shown). Despite the presence of CD36 on the viral membrane, the anti-CD36 mAb does not affect binding of the viral particles to the target cells and their infection. These antibody exposures did not have any effect on the cell viability (FIG. 6F).

Titration of the mAb revealed that as little as 32 ng/ml of mAb was sufficient to induce a reduction of the p24 release in the culture supernatant which was accompanied by an increase of the p24 level found in the corresponding cell lysates (FIGS. 7A and B).

The inhibitory effect of the antibody exposure was observed with two different mAbs reported to block uptake of LDL (Eugene A. Podrez et al, 1999, Eur J. Biochem, 774-784) (FIG. 6G), while antibodies specific for other scavenger receptors; CD68, SRB1, LDLR did not modify the amounts of p24 released by macrophages (FIG. 6H). Importantly, an anti-CD81 mAb (FIG. 6I), previously shown to be internalized into VCCs after 2 hours exposure at 37 did not modify the amounts of p24 released. Taken together these results established the high specificity of the inhibitory effect of the anti-CD36 mAbs. The inventors concluded that exposure of infected macrophages to the anti-CD36 mAb leads to the intracellular retention of viral particles and thus inhibit their release in the extracellular medium.

### Kinetic and Duration of the CD36-Specific mAb Effect on p24 Release by Infected Macrophages

The high specificity of the inhibitory effect of the anti-CD36 mAb and the relatively low doses required prompted us to better characterize this effect. First, the time period of mAb exposure was reduced (FIG. 8A, see timeline). The inhibitory effect was very clear after 8 hours but was detectable even after 2 hours (FIG. 8A). Finally, removal of the anti-CD36 mAb by acid washing after 2, 4 or 8 hours of exposure reduced but did not abolish the reduction of p24 released in the supernatant during the following 24 hours (FIG. 8B). This indicates that the effect induced was long lasting and confirmed that it did not result from the neutralization of the released virions but rather from an effect at the cellular level probable within the VCCs. The inventors conclude that exposure to anti-CD36 antibodies can be envisaged as a new biotherapy against HIV reservoirs.

### Specific Bivalent CD36 Antibodies Induce Rapid, Long-Lasting Inhibition of HIV-1 Release

VCCs as well as viral particles are enriched in many host proteins such as the CD81 tetraspanin and the ICAM-1 adhesion molecules. To evaluate whether the inhibition of virion release was restricted to CD36 specific antibodies, similar experiments were performed using other antibodies. Antibodies specific for CD81 and ICAM-1 accessed VCCs when added exogenously to infected macrophages at 37° C. for 2 hr (FIGS. 13A and B) but not at 4° C. (data not shown). However, exposure to antibodies specific for CD81, ICAM-1 or CD9 did not modify the amounts of p24 Gag released by macrophages (FIG. 13C to E), indicating that antibody access to the VCC was not sufficient to induce the retention of the virions. Similarly, exposure to antibodies specific for other lipid receptors, including scavenger receptors (CD68, SR-B1 or LDLR) did not modify the release of p24 as compared to their respective isotype controls (FIG. 13F).

Inhibition of HIV-1 release upon anti-CD36 mAb exposure remained unchanged in the presence of the FcR blocking reagent (FIG. 13G), indicating that Fc receptors were not involved in the antibody-mediated effect. Exposure of HIV-1-infected macrophages to the Fab fragment prepared from the CD36-specific CLB-ICV7 mAb (see comparative titration of the mAb and its Fab, FIG. 13H) at 3 concentrations (see arrows) did not affect p24 release, which was still inhibited by the bivalent mAb as expected (FIG. 13I). These results establish that the inhibitory effect of the anti-CD36 mAbs on p24 release by infected macrophages is highly specific, rapidly induced and requires bivalency.

Exposure to the Anti-CD36 Antibody Leads to Retention of Virions into VCCs

To evaluate the impact of the anti-CD36 mAb exposure on the HIV-1 cycle, the inventors looked at the maturation of the Gag precursor. The precursor p55Gag is synthesized as a cytosolic protein, which is cleaved once within the newly formed virions into 4 main polypeptides, including the Capsid p24. Exposure to the anti-CD36 mAb induced an intracellular accumulation of p24 Gag in infected macrophages as measured by Elisa (FIG. 14A). Quantification of the amounts of p55 and p24 Gag in cells from 7 donors was performed from immunoblot analyses. Results expressed as the amounts of p24, p55 or total Gag (FIG. 14B to D, respectively) normalized for the tubulin levels confirmed that anti-CD36 mAb exposure led to the accumulation of cell-associated p24 Gag that represents, in all likelihood, virions.

These results were confirmed by quantification of 3D reconstruction images of antibody-treated infected macrophages (see examples FIG. 14E). Different parameters were extracted including the sum of the Gag fluorescence intensity present in VCCs per cell (FIG. 14F) and the total volume of the VCCs per cell (FIG. 14G), which were both increased upon antibody exposure (statistically significant p≦0.001).

EM analysis of anti-CD36- and isotype-treated infected macrophages revealed that both types of cells possessed VCCs containing mature and immature virions (FIG. 14H). It was also observed budding profiles at the limiting membrane of the VCCs under both conditions suggesting that the antibody treatment did not affect the assembly process per se. Substantial amounts of anti-CD36 mAb were detected within VCCs of cells exposed to anti-CD36 mAb but not to the isotype control (FIG. 14I). Interestingly, the staining appeared rather associated to the membranes of the virions, suggesting that the mAb may create a network through its binding to the CD36 present in the viral membranes. The inventors conclude that the anti-CD36 mAb exposure induces the retention of virions within the VCCs of infected macrophages, increasing the volume of the VCCs and thus inhibiting the particle release to the extracellular medium.

### The MOxLDL-CD36 Pathway is not Activated by the Anti-CD36 mAb

Amongst the many ligands that CD36 can internalize in macrophages, MOxLDL has been widely studied. The effect of the anti-CD36 mAb could be related to the pathway induced by MOxLDL-CD36 interaction. To test this hypothesis, the inventors first exposed HIV-1-infected macrophages to freshly thawed MOxLDL or to the anti-CD36 mAb or its isotype control for 10 or 30 min and measured the phosphorylation of JNK. As expected, MOxLDL induced via CD36 the expected JNK phosphorylation (Rios et al., 2012) after 10 min of exposure (FIG. 15A) indicating that the MOxLDL used was biologically active. In contrast, anti-CD36 (or its isotype control) exposure had no effect on the levels of JNK phosphorylation, even after 30 min (FIG. 15A), suggesting that conventional signaling through CD36 does not take place upon exposure to the CD36 mAb.

To evaluate the impact of MOxLDL exposure on the virus release, HIV-1-infected macrophages were exposed to biologically active MOxLDL together or not with the anti-CD36 mAb or its isotype control for 24 hrs. In all cases the amount of p24 Gag measured in the supernatant was not modified by the presence of MOxLDL (FIG. 15B) nor was the cell viability (FIG. 15C).

The inventors also tested whether the anti-CD36 mAb-mediated inhibition of virion release was dependent on the presence of LDLs in the culture medium. HIV-1-infected macrophages were starved for 4 hrs and then cultured for 24 hrs in medium complemented with Lipoprotein Deficient Serum, or without any Fetal Calf Serum, and in the presence of the anti-CD36 mAb or its isotype control. In all cases the anti-CD36 mAb exposure led to a strong reduction in the amounts of p24 Gag secreted in the supernatant (FIG. 15D). These data indicate that exposure to the anti-CD36 mAb does not induce a signaling pathway similar to the one induced by MOxLDL. Moreover, these data rule out the possibility that the anti-CD36 mAb effect on particle release is linked to MOxLDL interaction with CD36. Besides MOxLDL, other endogenous ligands of CD36 include Trombospondin-1 and type-I Collagen. The inventors tested in a similar manner on infected macrophages whether overnight exposure to these two ligands exerts any effect on virion release. While exposure to the anti-CD36 mAb induced a strong inhibition, none of the two ligands reduced the release of p24 (FIG. 15E). They checked that both ligands were biologically active on uninfected macrophages by analyzing the phosphorylation status of the JNK and Src kinases (data not shown). Taken together these data suggest that the anti-CD36 mAb effect on particle release is not related to the pathways induced upon CD36 binding to some of its endogenous multivalent ligands such as MOxLDL, Trombospondin I or type-I Collagen.

Transfection of CD36 into HeLa Cells Promotes Appearance of Virus-Containing Compartments Upon HIV-1-Infection

HIV-1 assembly occurs into VCCs in macrophages and at the plasma membrane in many cell types and cell lines such as T lymphocytes and HeLa cells, respectively. The CD36 expression pattern is highly restricted to a few cell types including cells from the myeloid lineage (FIG. 11). Interestingly, T lymphocytes and HeLa cells do not express CD36. This suggested that CD36 may have a direct role on the location of the viral assembly process and the appearance of VCCs. To test this hypothesis, CD36-expressing HeLa cells were derived using a lentiviral-based approach (FIG. 9A) and infected with HIV-1 NL4-3. The inventors first evaluated the impact of CD36 expression on the amounts of p24 released. No significant difference was observed in the amount of p24 secreted and present in the lysates of control HeLa cells as compared to HeLa-CD36 (FIG. 9B). However, confocal microscopy analysis at day 2 post HIV-1 infection in HeLa cells revealed a Gag staining essentially cytosolic and diffuse with a few Gag+ dots observed at the plasma membrane level (FIG. 10). In contrast, in HIV-1-infected HeLa-CD36 cells, the inventors observed the presence of Gag+CD36+CD9+ intracellular compartments (FIG. 10). This suggested that expression of CD36 was sufficient to convert HeLa cells into a macrophage-like phenotype with the formation of VCCs upon HIV infection.

The inventors next tested the effect of the CD36 mAb in this cell system. Importantly, anti-CD36 mAb exposure of HIV-1-infected HeLa-CD36 cells but not regular HeLa cells led to a reduction of the amounts of p24 released which was accompanied by a concomitant increase of these levels in the cell lysates (FIG. 9C). This suggested that intracellular storage of viral particles occurs within CD36+ compartments that are sensitive to exposure to CD36-specific mAbs.

### Exposure of HIV-1-Infected HeLa-CD36 Cells to the Anti-CD36 mAb Induces Large Tethering of Viral Particles at the Plasma Membrane

To approach the mechanism of action of the anti-CD36 mAb, the inventors analyzed HIV-1-infected HeLa-CD36 cells exposed to the mAb for 24 hrs by confocal microscopy and observed the presence of large clusters of Gag+CD9+ material at the plasma membrane of cells exposed to the anti-CD36 mAb that were absent in isotype-treated cells (FIG. 16A). Time-lapse microscopy of HeLa-CD36 cells infected with HIV Gag-iGFP after addition of the anti-CD36 mAb allowed to witness the formation of these large clusters at the plasma membrane (data not shown). Ultrastructural analysis of HIV-1-infected HeLa-CD36 cells confirmed the presence at the plasma membrane of these large clusters of virions upon anti-CD36 exposure but not in the isotype treated control cells (FIG. 16B). Taken together these results suggest that the anti-CD36 exposure induces virions tethering at the site of viral assembly, i.e. in the VCCs of macrophages and at the plasma membrane of HeLa-CD36 cells.

### The C-Terminal Tail of CD36 is not Required for the Anti-CD36-Induced Inhibition of HIV Release.

These results suggested that the anti-CD36 mAb effect did not rely on signal transduction via CD36. To further test the hypothesis, the inventors took advantage of a previously published comparison of the cytoplasmic tail of CD36 with the ones of CD63, CD82, CD81, CD9 (Primo et al., 2005). All these proteins are present in the VCCs and present homologies (FIG. 16C) as they possess a bulky/aromatic residue (position 4), a Cys (position 5) and a basic residue (position 9). This suggested that potential motifs of targeting to the VCCs may exist in the cytoplasmic tail of CD36 as well as other VCC proteins.

To identify key residues involved in the anti-CD36-mediated effect, the inventors built a collection of CD36 mutants taking into account previous studies that have identified mutations altering several functions of CD36 (see FIG. 16D and references in the legend). Each of the 7 CD36 mutants as well as the CD36 wild type were cloned in a lentiviral vector that was used to establish stably expressing HeLa cells. All CD36 mutants were expressed at the cell surface as seen by flow cytometry with the exception of the CD36^{462*} mutant. Importantly, all the CD36 mutants expressed at the cell surface were sensitive to the inhibitory effect of the anti-CD36 mAb, like wild type CD36 (FIG. 16E). This includes point mutants of the conserved Cysteine residue (CD36^{C464s}) and a mutant carrying in its coding sequence a frame shift leading to substitution and premature stop codon (CD36^{shift}). These data suggest that the effect of the anti-CD36 mAb does not require the integrity of the CD36 cytoplasmic tail, and thus in all likelihood signal transduction through CD36 does not occur.

### CD36 mAb Inhibits the Release of a Primary Isolate and Macrophage to T Cell Viral Transmission

In a therapeutical perspective, the inventors evaluated if their results could be extended to primary strains of HIV-1. Anti-CD36 mAb exposure of macrophages infected with the 132W primary isolate (Roesch et al., 2012) led to a strong reduction of the p24 Gag secreted in the supernatant (FIG. 17A) and a concomitant increase of the cell-associated p24 Gag (FIG. 17B) without affecting cell viability (FIG. 17C).

Cell-to-cell transmission is thought to be much more efficient than virus-to-cell infection (Chen et al., 2007). To test the effect of the anti-CD36 mAb on macrophage-to-T-cell transmission, primary activated CD4+ T cells were added to HIV-1-infected macrophages together with the anti-CD36 mAb or its isotype control. After 24 hrs, AZT was added to stop new infections. The culture was left for an additional 24 hrs to allow Gag to accumulate into infected T cells, the proportion of which was measured by intracellular staining. As a positive control, anti-ICAM-1 mAb was used, which is known to inhibit cell-to-cell transfer of HIV-1 (Wang et al., 2009). Importantly, the anti-CD36 mAb exposure like the anti-ICAM-1 mAb but not the isotype control led to a strong reduction of the fraction of the T cells that became infected (FIG. 17D). These results were independently reproduced with cells from 3 donors (FIG. 17E). the inventors conclude that anti-CD36 mAb exposure of macrophages infected with a primary HIV-1 isolate leads to the inhibition of viral secretion and macrophage-to-T cell transmission.

Furthermore, this inhibitory effect appears virus-specific as the viral production of Influenza-infected macrophages remained unaffected by their exposure to the anti-CD36 mAb (data not shown).

### CONCLUSION

The inventors have herein demonstrated that HIV-1 hijacks pre-existing CD36+ compartments for viral assembly and storage. They showed that exposure to bivalent antibodies specific for CD36 induces the intracellular retention of the virions by infected macrophages and thus inhibits their release and transmission to T cells. They also demonstrated that silencing of CD36 inhibits the release of HIV-1 particles by infected macrophages.

Given the restricted pattern of CD36 expression among the various hematopoietic tissues, and the very high specificity of the effects, the inventors have documented that exposure to anti-CD36 antibodies can be envisaged as a new biotherapy against HIV reservoirs.

### REFERENCES

Benaroch, P., et al. (2010). Retrovirology 7, 29.
Bukrinsky, M., and Sviridov, D. (2006). J Leukoc Biol 80, 1044-1051.
Chen et al. 2007. Journal of virology. 81:12582-95.
Chertova, E., et al. (2006). J Virol 80, 9039-9052.
Collins, R. F., et al (2009). J Biol Chem 284, 30288-30297.
Crowe, S. M., et al (2010). J Leukoc Biol 87, 589-598.
Davies et al, Bio/Technology, 13, 475-479 (1995).
Deneka, M., et al (2007). J Cell Biol 177, 329-341.
Gaudin et al. 2012. The Journal of cell biology. 199:467-79.
Hamers-Casterman et al., 1993. Nature, 1993, 363:446
Hsu, N.-Y., et al. (2010). Cell 141, 799-811.
Jouve, 2007 Cell Host Microbe 2(2):85-95
Kruth, H. S., et al (1999). J Biol Chem 274, 7495-7500.
Kruth, H. S., et al (1995). J Cell Biol 129, 133-145.
Lo, J., et al (2010). AIDS 24, 243-253.
Manel, 2010, Nature 467 (7312):214-7
Micheletti, R. G., et al (2009). Cardiovasc. Pathol. 18, 28-36.
Moore, K. J., and Freeman, M. W. (2006). Arterioscler. Thromb. Vasc. Biol. 26, 1702-1711.
Morner, 1999 Virus Res 59(1): 49-60
Mujawar, Z., et al. (2006). PLoS Biol 4, e365.
Pelchen-Matthews, A., et al. (2011). Traffic 13(2):273-91
Primo et al., 2005, The FASEB Journal 19, 1713-1715
Rasheed, S., et al. (2008). PLoS ONE 3, e3003.
Rios et al. 2012. PloS one. 7:e36632.
Roesch et al. 2012. PLoS pathogens. 8:e1002792.
Wang et al. 2009. Journal of virology. 83:4195-204.
Welsch, S., et al (2007). PLoS Pathog 3, e36.
Zeng, Y., et al (2003). J Biol Chem 278, 45931-45936.

1-42. (canceled)

43. A method of treating a subject with an HIV infection comprising administering a composition comprising a pharmaceutically acceptable excipient and an antibody that recognizes and binds CD36, or an antigen-binding fragment or derivative thereof, a CD36 antagonist, or a conjugate comprising at least one CD36 targeting agent associated to at least one anti-HIV effector moiety to a subject having an HIV infection.

44. The method according to claim 43, wherein said composition comprises a CD36 antagonist.

45. The method according to claim 43, wherein the CD36 antagonist is selected from the group consisting of an antibody or an aptamer which specifically recognizes and binds to CD36 or a fragment thereof, a nucleic acid molecule interfering specifically with CD36 expression, and a small molecule inhibiting the CD36 activity.

46. The method according to claim 43, wherein the CD36 antagonist is a function-blocking monoclonal antibody directed against CD36 or a nucleic acid molecule interfering specifically with CD36 expression.

47. The method according to claim 43, wherein the CD36 antagonist is a nucleic acid molecule interfering specifically with CD36 expression.

48. The method according to claim 43, wherein the CD36 antagonist is a nucleic acid molecule interfering specifically with CD36 expression selected from the group consisting of a RNAi, an antisense nucleic acid and a ribozyme.

49. The method according to claim 43, wherein the CD36 antagonist is a small molecule selected from the group consisting of salvianolic acid B, rosmarinic acid, sodium danshensu, 3-cinnamoyl indole, 13 pentyl berberine, hexarelin, nanoblockers, statins or antioxidants such as α-tocopherol and SS peptides, Sulfo-N-succinimidyl oleate and Ursolic acid, and any combination thereof.

50. The method according to claim 43, wherein said composition comprises an antibody that recognizes and binds CD36, or an antigen-binding fragment or derivative thereof.

51. The method according to claim 50, wherein said antibody or antigen-binding fragment or derivative thereof, is directed against the extracellular domain of CD36.

52. The method according to claim 50, wherein said antibody is a full-length antibody.

53. The method according to claim 50, wherein the antibody is a monoclonal antibody.

54. The method according to claim 50, wherein the antibody is of the IgG, IgE or IgD type.

55. The method according to claim 50, wherein the antibody is a humanized, chimeric or human antibody.

56. The method according to claim 50, wherein said antibody, or antigen-binding fragment or derivative thereof, is bivalent.

57. The method according to claim 50, wherein the antigen-binding fragment is selected from the group consisting of F(ab′)₂, di-scFvs, sc(Fv)₂, (VHH)₂ fragment and a diabody.

58. The method according to claim 43, wherein the composition comprises a conjugate comprising at least one CD36 targeting agent associated to at least one anti-HIV effector moiety.

59. The method according to claim 58, wherein said CD36 targeting agent is an antibody recognizing and binding to CD36, or to a fragment thereof.

60. The method according to claim 58, wherein said anti-HIV effector moiety is a HIV co-receptor or an anti-HIV drug.

61. The method according to claim 60, wherein said anti-HIV effector moiety is a HIV co-receptor selected from the group consisting of CXCR4, CCR5, CD4, T20, NBD-556, CD4M33, fragments thereof and functional equivalents thereof.

62. The method according to claim 60, wherein said anti-HIV effector moiety is an anti-HIV drug selected from the group consisting of reverse transcriptase inhibitors, integrase inhibitors and protease inhibitors.

63. The method according to claim 43, wherein said composition is administered in combination with another anti-viral treatment.

64. The method according to claim 63, wherein said another anti-viral treatment is highly active retroviral therapy (HAART).

65. The method according to claim 64, wherein the highly active retroviral therapy comprises a combination of one or several reverse transcriptase inhibitors, one or more integrase inhibitors, and/or one or several protease inhibitors.

66. The method according to claim 64, wherein the composition is administered before, during or after said another anti-viral treatment.

67. A conjugate comprising at least one CD36 targeting agent associated to at least one anti-HIV effector moiety.

68. The conjugate according to claim 67, wherein said CD36 targeting agent is an antibody recognizing and binding to CD36, or to a fragment thereof.

69. The conjugate according to claim 67, wherein said anti-HIV effector moiety is a HIV co-receptor or an anti-HIV drug.

70. The conjugate according to claim 69, wherein said anti-HIV effector moiety is a HIV co-receptor selected from the group consisting of CXCR4, CCR5, CD4, T20, NBD-556, CD4M33, fragments thereof and functional equivalents thereof.

71. The conjugate according to claim 69, wherein said anti-HIV effector moiety is an anti-HIV drug selected from the group consisting of reverse transcriptase inhibitors, integrase inhibitors and protease inhibitors.

72. A method for screening for compounds useful for the treatment of HIV infection, said method comprising determining whether a candidate compound recognizes and binds to CD36 or reduces or inhibits expression of CD36, wherein the ability of said candidate compound to recognize and bind to CD36 or to reduce or inhibit expression of CD36 is indicative of the usefulness of said candidate for the treatment of HIV infection.


SEQUENCE LISTING (9 sequences)

SEQ ID NO: 1 - DNA, 21 bp, Artificial Sequence
  siCD36_1
gaaccuauugauggauuaatt

SEQ ID NO: 2 - DNA, 21 bp, Artificial Sequence
  siCD36_3
ccuucacuaucaguuggaatt

SEQ ID NO: 3 - DNA, 21 bp, Artificial Sequence
  siCD36_4
gcaacauucaaguuaagcatt

SEQ ID NO: 4 - DNA, 31 bp, artificial
  primer
tgtacaaacgcgtgtgagcaagggcgaggag

SEQ ID NO: 5 - DNA, 32 bp, artificial
  primer
ttggcttctagacttgtacagctcgtccatgc

SEQ ID NO: 6 - DNA, 36 bp, artificial
  primer
accgcgggcccgggatatgggctgtgaccggaactg

SEQ ID NO: 7 - DNA, 44 bp, artificial
  primer
catggtggcgaccggtggttttattgttttcgatctgcatgcac

SEQ ID NO: 8 - DNA, 21 bp, artificial
  Luc
cguacgcggaauacuucgatt

SEQ ID NO: 9 - RNA, 21 nt, artificial
  Tsg101
caguuuaucauucaaguguaa